(19) United States
(12) Patent Application Publication
(10) Pub. No.: US 2024/0194298 A1
(43) Pub. Date: Jun. 13, 2024

(54) METHOD FOR CLASSIFYING QUALITY OF BIOLOGICAL SENSOR DATA

(71) Applicant: Hoffmann-La Roche Inc., Little Falls, NJ (US)

(72) Inventors: Lito Kriara, Basel (CH); Florian Lipsmeier, Basel (CH); Mattia Zanon, Basel (CH)

(21) Appl. No.: 18/581,036

(22) Filed: Feb. 19, 2024

Related U.S. Application Data
(63) Continuation of application No. PCT/EP2022/073126, filed on Aug. 18, 2022.

(30) Foreign Application Priority Data
Aug. 19, 2021 (EP) 21192193.7

(51) Int. Cl. G16B 40/10; G16B 40/20

(52) U.S. Cl. CPC G16B 40/10; G16B 40/20

(57) ABSTRACT

A computer implemented method for classifying quality of biological sensor data (110) comprising the following steps:
providing biological sensor data (110) obtained by at least one biological sensor (112), wherein the biological sensor data (110) comprises at least one signal (116);
classifying quality of the signal (116) by using at least one trained trainable model (119), wherein the trainable model (119) is trained on historical biological sensor data based on a supervised (134) and/or semi-supervised deep learning architecture (188), wherein the trainable model (119) is trained by optimizing one loss function in terms of classification or two loss functions in terms of signal reconstruction and classification.

Further, a biological sensor (112), a computer program and a computer-readable storage medium are disclosed.

### TECHNICAL FIELD

The present invention refers to a computer implemented method for classifying quality of biological sensor data. The invention further relates to a biological sensor and to a computer program and a computer-readable storage medium for performing the method according to the present invention. The method and devices, in particular, may be used in the field of body worn devices such as wrist-worn devices or head-worn devices. For example, the biological sensor may be worn on the wrist or head for example. Other measurement positions, however, are possible such as chest or finger. Other fields of application of the present invention, however, are feasible.

### BACKGROUND ART

Wearable sensors are broadly used for collecting physiological and behavioral signals, used for health monitoring and even as medical devices, as described in Coravos, A., Khozin, S., and Mandl, K. D., “Developing and adopting safe and effective digital biomarkers to improve patient outcomes”, npj Digital Medicine, 2(14), 2019. Predictions of these health monitoring tools or medical devices are only as reliable as the sensor data used. Sensor data quality may depend on hardware and can be highly prone to noise. Therefore, the signal quality and actual feature estimates have been shown to vary, as described in Sequeira, N. et al., “Common wearable devices demonstrate variable accuracy in measuring heart rate during supraventricular tachycardia, Heart Rhythm, 17(5), 2020 and Pasadyn, S. R., et al., “Accuracy of commercially available heart rate monitors in athletes: A prospective study. Cardiovascular Diagnosis and Therapy”, 9(4):379-385, 2019. In order to be able to make reliable predictions or assumptions regarding the wellbeing of the person carrying the sensor device, it may be necessary to be confident that only using reliable data or signals are used. The signal quality of sensors may be negatively influenced by factors such as motion artifacts, sensor placement, and even blood perfusion or skin type, e.g., photoplethysmograph (PPG), electrocardiogram (ECG), electroencephalogram (EEG), e.g as described in Bent, B., et al., “Investigating sources of inaccuracy in wearable optical heart rate sensors”. npj Digital Medicine, 3(18), 2020.

Thus, there is a need to have a reliable classification of clean vs noisy signals, which currently does not exist as a standard methodology. Instead, for different applications people tend to use sensor-specific heuristics, see e.g. Bhowmik, T., et al., “A novel method for accurate estimation of HRV from smartwatch PPG signals”, in IEEE Engineering in Medicine and Biology Society, pp. 109-112, 2017, or methodologies that report a continuous quality index, which then poses the non-straightforward question “what should be the signal quality threshold to label a signal as clean or noisy” as described in Orphanidou, C., et al., “Signal-quality indices for the electrocardiogram and photoplethysmogram: Derivation

and applications to wireless monitoring”, IEEE Journal of Biomedical and Health Informatics, 19(3):832-838, 2014, Elgendi, M., “Optimal signal quality index for photoplethysmogram signals”, Scientific Reports, 3(4), 2016, and Zanon, M., et al., “A quality metric for heart rate variability from photoplethysmogram sensor data”, in IEEE Engineering in Medicine and Biology Society, pp. 706-709, 2020.

US 2019/133468 A1 describes an apparatus which includes a sensor module, a data processing module, a quality assessment module and an event prediction module. The sensor module provides biosignal data samples and motion data samples. The data processing module processes the biosignal data samples to remove baseline and processes the motion data samples to generate a motion significant measure. The quality assessment module generates a signal quality indicator based on the processed biosignal data sample segments and the corresponding motion significance measure using a first deep learning model. The event prediction module generates an event prediction result based on the processed biosignal data sample segments associated with a desired signal quality indicator using a second deep learning model.

### Problem to be Solved

It is therefore desirable to provide methods and devices which address the above-mentioned technical challenges of using wearable sensors for health monitoring and/or as medical devices. Specifically, methods and devices shall be proposed which overcome the need for extensive manual annotations.

### SUMMARY

This problem is addressed by a computer implemented method for classifying quality of biological sensor data with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the invention, a computer implemented method for classifying quality of biological sensor data is disclosed.

The term “computer implemented method” as used herein is a broad term and is to be given its ordinary and customary meaning to a person of ordinary skill in the art and is not to be limited to a special or customized meaning. The term specifically may refer, without limitation, to a method involving at least one computer and/or at least one computer network or a cloud. The computer and/or computer network and/or a cloud may comprise at least one processor which is configured for performing at least one of the method steps of the method according to the present invention. Preferably each of the method steps is performed by the computer and/or computer network and/or a cloud. The method may be performed completely automatically, specifically without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or a cloud and/or machine, in particular without manual action and/or interaction with a user.

The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlap-ping fashion. The method may comprise further method steps which are not listed.

The method comprises the following steps:
a) providing biological sensor data obtained by at least one biological sensor, wherein the biological sensor data comprises at least one signal;
b) classifying quality of the signal by using at least one trained trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.

The term “biological sensor” as used herein is a broad term and is to be given its ordinary and customary meaning to a person of ordinary skill in the art and is not to be limited to a special or customized meaning. The term specifically may refer, without limitation, to an arbitrary device configured for one or more of detecting, measuring or monitoring at least one biological measurement variable or biological measurement property. Specifically, the biological sensor may be capable of generating at least one signal, such as a measurement signal, which is a qualitative or quantitative indicator of the measurement variable and/or measurement property. The biological sensor may be configured for qualitatively and/or quantitatively determining at least one health condition and/or at least one measurement variable indicative of a health condition of a subject. The term “subject” as used herein refers to an animal, preferably a mammal and, more typically to a human. The biological sensor may be configured for detecting and/or measuring either quantitatively or qualitatively at least one biological and/or physical and/or chemical parameter of the subject and for transforming the detected and/or measured parameter into at least one signal such as for further processing and/or analysis.

The biological sensor may be a portable, in particular handheld and/or wearable, biological sensor. The term “portable” as used herein is a broad term and is to be given its ordinary and customary meaning to a person of ordinary skill in the art and is not to be limited to a special or customized meaning. The term specifically may refer, without limitation, to a property of the biological sensor allowing that a user can one or more of hold and/or wear and/or transport the biological sensor. Specifically, the biological sensor may be wearable. For example, the biological sensor may be a wristwatch such as a smartwatch. Other measurement positions, however, are possible such as or head, chest or finger. Using a portable biological sensor may result in that disturbances can influence the measurement such as motions artefacts. Uncontrolled conditions met in daily life may pose several challenges related to disturbances that can deteriorate the signal making the determination of the health condition untrustworthy and not reliable.

The biological sensor may be or may comprise one or more of at least one photoplethysmogram (PPG) device, at least one electrocardiogram (ECG) device, at least one electroencephalogram (EEG) device. However, other biological sensors are feasible.

For example, the biological sensor may be at least one portable photoplethysmogram device. The biological sensor data may comprise at least one photoplethysmogram obtained by the portable photoplethysmogram device. The term “photoplethysmogram device” as used herein is a broad term and is to be given its ordinary and customary meaning to a person of ordinary skill in the art and is not to be limited to a special or customized meaning. The term specifically may refer, without limitation, to at least one device configured for determining at least one photoplethysmogram. The term “plethysmogram” as used herein is a broad term and is to be given its ordinary and customary meaning to a person of ordinary skill in the art and is not to be limited to a special or customized meaning. The term specifically may refer, without limitation, to a result of a measurement of volume changes of at least one part of the human body or of organs. The term “photoplethysmogram” (PPG) as used herein is a broad term and is to be given its ordinary and customary meaning to a person of ordinary skill in the art and is not to be limited to a special or customized meaning. The term specifically may refer, without limitation, to an optically determined plethysmogram. The PPG may show development of a signal from the PPG device over time.

The photoplethysmogram device may comprise at least one illumination source. The term “illumination source” as used herein is a broad term and is to be given its ordinary and customary meaning to a person of ordinary skill in the art and is not to be limited to a special or customized meaning. The term specifically may refer, without limitation, to at least one arbitrary device configured for generating at least one light beam. The illumination source may comprise at least one light source such as at least one light-emitting-diode (LED) transmitter. The illumination source may be configured for generating at least one light beam for illuminating e.g. the skin on at least one part of the human body. The illumination source may be configured for generating light in the red, infrared or green spectral region. As used herein, the term “light”, generally, refers to a partition of electromagnetic radiation which is, usually, referred to as “optical spectral range” and which comprises one or more of the visible spectral range, the ultraviolet spectral range and the infrared spectral range. Herein, the term “ultraviolet spectral range”, generally, refers to electromagnetic radiation having a wavelength of 1 nm to 380 nm, preferably of 100 nm to 380 nm. The term “visible spectral range”, generally, refers to a spectral range of 380 nm to 760 nm. The term “infrared spectral range” (IR) generally refers to electromagnetic radiation of 760 nm to 1000 μm, wherein the range of 760 nm to 1.5 μm is usually denominated as “near infrared spectral range” (NIR) while the range from 1.5μ to 15 μm is denoted as “mid infrared spectral range” (MidIR) and the range from 15 μm to 1000 μm as “far infrared spectral range” (FIR).

The photoplethysmogram device may comprise at least one photodetector, in particular at least one photosensitive diode. The term “photodetector” as used herein is a broad term and is to be given its ordinary and customary meaning to a person of ordinary skill in the art and is not to be limited to a special or customized meaning. The term specifically may refer, without limitation, to at least one light-sensitive device for detecting a light beam, such as for detecting an illumination generated by at least one light beam. The photodetector may be configured for detecting light from transmissive absorption and/or reflection in response to illumination by the light generated by the illumination source.

The PPG device may be configured for measuring blood volume variations due to heartbeat by shining light into the skin and measuring the light that is reflected back. With respect to design of a PPG device reference is made to Biswas, D., et al., “Heart rate estimation from wrist-worn photoplethysmography: A review”, IEEE Sensors Journal, 19(16):6560-6570, 2019. Specifically, the PPG may represent an aggregated expression of many physiological processes within the cardiovascular system as described in Liang, Y., et al.: “An optimal filter for short photoplethysmogram signals”, Scientific Data, 5(180076), 2018. When the PPG signal is reliable, it may be possible to compute heart rate (HR) and heart rate variability (HRV) features, e.g. in order to understand multiple aspects of a person's physical, psychological and mental state, like exercise recovery, see e.g. Bechke, E., et al., “An examination of single day vs. multi-day heart rate variability and its relationship to heart rate recovery following maximal aerobic exercise in females”, Scientific Reports, 10(14760), 2020, cardio conditions, see e.g. Hoshi, R. A., et al., Reduced heart-rate variability and increased risk of hypertension-a prospective study of the elsa-brasil. Journal of Human Hypertension, 2021, sleeping patterns, see e.g. Hictakoste, S., et al., “Longer apneas and hypopneas are associated with greater ultra-short-term hrv in obstructive sleep apnea”, Scientific Reports, 10(21556), 2020, anxiety, see e.g. Rodrigues, J., et al., “Locomotion in virtual environments predicts cardiovascular responsiveness to subsequent stressful challenges”, Nature Communications, 11(5904), 2020, and emotional state, see e.g. Kim, J. J., et al. “Neurophysiological and behavioral markers of compassion”, Scientific Reports, 10(6789), 2020.

The term “biological sensor data” as used herein is a broad term and is to be given its ordinary and customary meaning to a person of ordinary skill in the art and is not to be limited to a special or customized meaning. The term specifically may refer, without limitation, to data obtained via the biological sensor such as measurement data. The biological sensor data comprises at least one signal, also denoted as sensor signal. The term “signal” as used herein is a broad term and is to be given its ordinary and customary meaning to a person of ordinary skill in the art and is not to be limited to a special or customized meaning. The term specifically may refer, without limitation, to at least one electrical signal, such as at least one analogue electrical signal and/or at least one digital electrical signal. More specifically, the sensor signal may be or may comprise at least one voltage signal and/or at least one current signal. More specifically, the sensor signal may comprise at least one photocurrent. For example, the signal may be at least one electronic signal of the PPG device, in particular of the photodetector, depending on detected light from transmissive absorption and/or reflection in response to illumination by the light generated by the illumination source.

Further, either raw signals may be used, or processed or preprocessed signals may be used, thereby generating secondary signals, which may also be used as sensor signals. The method may comprise at least one pre-processing step comprising one or more of filtering or normalizing the biological sensor data. For example, in case of a signal of the PPG device a bandpass filter may be used. Additionally, the signal may be normalized so that the values are around 0. However, preprocessing can be different for different signals depending on the physiology.

For example, the signal may be a PPG signal. PPG signals can be easily extracted from human peripheral tissue, such as fingers, toes, earlobes, wrists, and the forehead. Therefore, they may have great potential for application in wearable health devices, as described e.g. in Liang et al. For example, the PPG signals may be collected via a smartwatch, in particular a smartwatch on the wrist equipped with LEDs and photodiode. Generally, arbitrary sampling frequency is possible. High sampling frequency may be preferred. For example, the photoplethysmogram device, e.g. the smartwatch, may be configured for measuring a PPG at 20 Hz sampling frequency. For example, the photoplethysmogram device may be configured measuring a PPG with a frequency from 20 Hz to 1 kHz. The smartwatch may be custom smartwatch, e.g. a Samsung Gear® Sport smartwatch. The PPG signals may be pre-processed using a third order Butterworth bandpass filter with 0.5 and 9 Hz frequency cut on per subject daily PPG signals. The daily PPG signal may be cut into intervals, e.g. 10 second intervals.

The term “providing” as used herein is a broad term and is to be given its ordinary and customary meaning to a person of ordinary skill in the art and is not to be limited to a special or customized meaning. The term specifically may refer, without limitation, to measuring the biological sensor data and/or retrieving the biological sensor data. The term “retrieving” as used herein is a broad term and is to be given its ordinary and customary meaning to a person of ordinary skill in the art and is not to be limited to a special or customized meaning. The term specifically may refer, without limitation, to a process of a system specifically a computer system, of generating data and/or obtaining data from the biological sensor and/or a data storage, e.g. from a network or from a further computer or computer system. The retrieving specifically may take place by at least one computer interface, such as via a port such as a serial or parallel port. The retrieving may comprise several sub-steps, such as the sub-step of obtaining one or more items of primary information and generating secondary information by making use of the primary information, such as by applying one or more algorithms to the primary information, e.g. by using a processor.

The term “quality”, also denoted as signal quality, as used herein is a broad term and is to be given its ordinary and customary meaning to a person of ordinary skill in the art and is not to be limited to a special or customized meaning. The term specifically may refer, without limitation, to a measure for reliability of a signal determined by the biological sensor. Specifically, the quality may be classified as good for reliable signals and as bad for non-reliable signals. The classifying of quality may comprise discriminating between noisy and clean signals. The quality may be classified dependent on presence of noise and/or artifacts. The reliability of the signal may decrease with increasing noise and/or artifacts. The quality may be negatively influenced by a plurality of factors such as motion artifacts, sensor placement, blood perfusion and/or skin type.

The quality may be used as quality indicator for heart rate variability data. The term “heart rate variability” (HRV) as used herein is a broad term and is to be given its ordinary and customary meaning to a person of ordinary skill in the art and is not to be limited to a special or customized meaning. The term specifically may refer, without limitation, to a measure of regularity between consecutive heartbeats. The quality may be used for distinguishing between acceptable and non-acceptable heart rate variability data.

The quality of the obtained biological sensor data may be provided to a user, such as the subject, via at least one user-interface. The term “user interface” as used herein is a broad term and is to be given its ordinary and customary meaning to a person of ordinary skill in the art and is not to be limited to a special or customized meaning. The term may refer, without limitation, to an element configured for interacting with its environment, such as for the purpose of unidirectionally or bidirectionally exchanging information, such as for exchange of one or more of data or commands. For example, the user interface of the smartwatch may be configured to share information with a user and to receive information by the user. The user interface may be designed to interact visually with a user, such as a display, and/or to interact acoustically with the user. The user interface, as an example, may comprise one or more of: a graphical user interface; a data interface, such as a wireless and/or a wire-bound data interface. Thus, the provided quality may be used for interpreting biological sensor data obtained by the biological sensor. Additionally or alternatively, the biological sensor, such as the smartwatch, may comprise at least one controlling unit configured for dismissing and/or rejecting biological sensor data categorized as noisy or bad quality.

The term “classifying” as used herein is a broad term and is to be given its ordinary and customary meaning to a person of ordinary skill in the art and is not to be limited to a special or customized meaning. The term specifically may refer, without limitation, to a process of categorizing the signal into at least two categories, such as noisy or clean signal.

Classifying quality of the signal is performed by using at least one trained trainable model. The term “trainable model” as used herein is a broad term and is to be given its ordinary and customary meaning to a person of ordinary skill in the art and is not to be limited to a special or customized meaning. The term specifically may refer, without limitation, to a mathematical model which is trainable on at least one training dataset using one or more of machine learning, in particular deep learning or other form of artificial intelligence. The term “machine learning” as used herein is a broad term and is to be given its ordinary and customary meaning to a person of ordinary skill in the art and is not to be limited to a special or customized meaning. The term specifically may refer, without limitation, to a method of using artificial intelligence (AI) for automatically model building. The term “deep learning” as used herein is a broad term and is to be given its ordinary and customary meaning to a person of ordinary skill in the art and is not to be limited to a special or customized meaning. The term specifically may refer, without limitation, to a class of machine learning algorithms using multiple layers, in particular using deep learning architectures such as one or more of deep neural networks, deep belief networks, graph neural networks, recurrent neural networks and convolutional neural networks. For example, the trainable model may comprise at least one deep neural network selected from the group consisting of: Convolutional Neural Network (CNN) layers such as in the WaveNet architecture, a recurrent neural network (RNN), a Long short-term memory (LSTM). For example, an architecture inspired by the WaveNet architecture may be used. With respect to WaveNet reference is made to van den Oord, et al., “Wavenet: A generative model for raw audio”, CoRR, abs/1609.03499, 2016. The deep neural network may use stacked causal dilated convolutions. Using a WaveNet-like architecture on PPG data is a novel and unique approach. The skilled person would not use a WaveNet-like architecture because it was originally developed for using it on speech data, and thus, for a very different data type. However, it was surprisingly found that using WaveNet-like architecture on PPG data allows for classifying quality of PPG data with increased reliability.

The training may be performed using at least one machine-learning system. The term “machine-learning system” as used herein is a broad term and is to be given its ordinary and customary meaning to a person of ordinary skill in the art and is not to be limited to a special or customized meaning. The term specifically may refer, without limitation, to a system or unit comprising at least one processing unit such as a processor, microprocessor, or computer system configured for machine learning, in particular for executing a logic in a given algorithm. The machine-learning system may be configured for performing and/or executing at least one machine-learning algorithm, wherein the machine-learning algorithm is configured for building the trained trainable model. The machine-learning system may be part of the biological sensor and/or may be performed by an external processor.

The term “training” as used herein is a broad term and is to be given its ordinary and customary meaning to a person of ordinary skill in the art and is not to be limited to a special or customized meaning. The term specifically may refer, without limitation, to a process of building the trained trainable model, in particular determining parameters, in particular weights, of the model. The training may comprise determining and/or updating parameters of the model. The trained trainable model may be at least partially data driven. As used herein, the term “at least partially data-driven model” is a broad term and is to be given its ordinary and customary meaning to a person of ordinary skill in the art and is not to be limited to a special or customized meaning. The term specifically may refer, without limitation, to the fact that the model comprises data-driven model parts and other model parts based on physico-chemical laws. The training may be performed on biological sensor data. The training may comprise retraining a trained trainable model, e.g. after obtaining additional biological sensor data such as during wearing and operating the smartwatch.

The trainable model is trained on historical biological sensor data based on a supervised and/or semi-supervised deep learning architecture. The term “historical biological sensor data” as used herein is a broad term and is to be given its ordinary and customary meaning to a person of ordinary skill in the art and is not to be limited to a special or customized meaning. The term specifically may refer, without limitation, to at least one independent data set used for training of the deep learning architecture. The historical biological sensor data is independent from the patient, runtime or test data.

The method further may comprise:
c) at least one training step, wherein, in the training step, the trainable model is trained on at least one training dataset comprising the historical biological sensor data, based on the supervised and/or semi-supervised deep learning architecture, wherein the trainable model is trained by optimizing the one loss function in terms of classification or the two loss functions in terms of signal reconstruction and classification.

The trainable model based on the supervised deep learning architecture may also be denoted as supervised model herein. The trainable model based on the semi-supervised deep learning architecture may also be denoted as semi-supervised model herein.

The term “supervised” deep learning architecture as used herein is a broad term and is to be given its ordinary and customary meaning to a person of ordinary skill in the art and is not to be limited to a special or customized meaning. The term specifically may refer, without limitation, to a deep learning architecture learning based on labeled historical biological sensor data. In particular, manual labeled historical biological sensor data may be used for training the trainable model based on the supervised deep learning architecture. For example, as historical biological sensor data, a manual labeled PPG dataset may be used. For example, a training dataset of biological sensor data may be set up as follows: Data was collected from 5 healthy volunteers (1 female and 4 male with average age of 33) without any supervision, during their normal daily activities, or during their night sleep. In total 13547 non-overlapping PPG signal samples were collected, of 10 seconds length each. The signals may be manually labeled by experts according to the instructions of Elgendi, M., “Optimal signal quality index for photoplethysmogram signals”, Scientific Reports, 3(4), 2016. 8305 noisy, and 5242 clean PPG signals were categorized. For example, a balanced dataset of 9380 labeled signal samples may be used as training dataset.

The training step may comprise preprocessing the historical biological sensor data, e.g. filtering the PPG signals of 10 seconds each with 20 Hz frequency, with in total 200 data points. The labels may be provided for each input signal for training with “0” indicating a noisy and “1” a clean signal.

The supervised deep learning architecture may comprise at least one input layer receiving the historical biosensor data and/or preprocessed historical biosensor data. For example, as input filtered PPG signals of 10 seconds each with 20 Hz frequency may be used. Thus, the input may comprise a signal comprising 200 values. With different sampling frequencies or lengths in seconds, the values of the PPG signal would vary.

The supervised deep learning architecture may comprise a plurality of convolutional layers, in particular a stack of convolutional layers. For example, the supervised deep learning architecture may comprise five convolutional layers. The convolutional layers may be designed with dilation. The convolutional layers may be configured for dilated convolution. The supervised deep learning architecture may comprise a WaveNet-like neural network architecture. As e.g. described in van den Oord, et al., “Wavenet: A generative model for raw audio”, CoRR, abs/1609.03499, 2016, the main ingredient of WaveNet may be causal convolutions. By using causal convolutions, it may be possible to ensure that the deep learning architecture cannot violate an ordering in which the data is modeled, in particular cannot depend on any of the future time steps. The deep learning architecture having causal convolutions may not have recurrent connections, such that they are typically faster to train than RNNs, especially when applied to very long sequences. One of the problems of causal convolutions, however, may be that they require many layers, or large filters to increase the receptive field. Therefore, WaveNet-like architectures may use dilated convolutions to increase the receptive field by orders of magnitude, without greatly increasing computational cost. The term “dilated convolution” as used herein is a broad term and is to be given its ordinary and customary meaning to a person of ordinary skill in the art and is not to be limited to a special or customized meaning. The term specifically may refer, without limitation, to a convolution where a filter is applied over an area larger than its length by skipping input values with a certain step. It may be equivalent to a convolution with a larger filter derived from the original filter by dilating it with zeros, but may be significantly more efficient. A dilated convolution effectively may allow the network to operate on a coarser scale than with a normal convolution. This may be similar to pooling or strided convolutions, but here the output may have the same size as the input. In particular, the stacked convolutional layers allowing stacked dilated convolutions may enable the network to have very large receptive fields with just a few layers, while preserving the input resolution throughout the network as well as computational efficiency.

The supervised deep learning architecture may comprise causal padding in each convolutional layer.

The supervised deep learning architecture may comprise at least one flatten layer after the convolutional layers and before the outputs. The flatten layer may be designed to transform a matrix output of the convolutional layers into a dense layer. A dense layer may be a neural network structure in which all neurons are connected to all inputs and all outputs.

The supervised deep learning architecture may comprise at least one optimizer, in particular an Adam optimizer. With respect to Adam optimizer reference is made to Diederik P. Kingma, Jimmy Ba, “Adam: A Method for Stochastic Optimization”, 3rd International Conference for Learning Representations, San Diego, 2015.

For example, the supervised deep learning architecture may comprise five convolutional layers. The first layer may have no dilation, the second one a dilation of 2, and from there on dilation may double for each next layer. In each layer 16 filters may be used. A kernel of size 3, 5, 7 or even other sizes may be used. A regularization strength may be in the range of 0.0005 and 0.002, e.g. 0.0005, 0.001, 0.0015 or 0.002. However, other ranges are possible. The supervised deep learning architecture may comprise a flatten layer after the convolutional layers and before the outputs. The supervised deep learning architecture may comprise an Adam optimizer, e.g. with learning rate of 0.00001 and with a decay where the learning rate is halved every ten or 100 or more epochs. However, other learning rates and learning decay rates are possible. For example, for training, a batch size of 128, and epochs up to 300 may be used. For example, for training, a batch size of 128, and epochs from 50 to 500 or even more may be used. However, other batch size and epochs are possible.

The deep learning architecture may comprise as final layer, in particular a dense layer, an output layer comprising two paths. Each of the paths may comprise an output. Specifically, the deep learning architecture may comprise two outputs, a first and a second output. The trainable model is trained by optimizing one loss function in terms of classification or two loss functions in terms of signal reconstruction and classification. The supervised deep learning architecture may be trained by optimizing one loss function in terms of classification. The supervised deep learning architecture may be trained by optimizing two loss functions in terms of signal reconstruction and classification. The semi-supervised deep learning architecture may be trained by optimizing two loss functions in terms of signal reconstruction and classification. The term “loss function” as used herein is a broad term and is to be given its ordinary and customary meaning to a person of ordinary skill in the art and is not to be limited to a special or customized meaning. The term specifically may refer, without limitation, to a function that assigns to each decision, in the form of a point estimate, a range estimate, or a test, the loss that results from a decision deviating from the true parameter. The training of the trainable model may comprise solving an optimization problem, in particular optimizing the loss functions. The term “optimizing a loss function” as used herein is a broad term and is to be given its ordinary and customary meaning to a person of ordinary skill in the art and is not to be limited to a special or customized meaning. The term specifically may refer, without limitation, to a process of minimizing the loss function. The trainable model may be trained by optimizing a first loss functions in terms of classification, or the first loss function and a second loss function in terms of signal reconstruction.

The first output may be a class output providing the classified quality. The class output may use the first loss function. The first loss function may relate to classification loss. For example, the class output may take the flatten or dense layer's output as input. The class output may use a sigmoid activation function. The class output may use a binary crossentropy loss function to provide a probability between 0 and 1, with a value over 0.5 indicating that the signal is clean.

The second output may be a mean squared error (MSE) output providing a measure for a difference between the input and reconstructed signal after the convolutions. The MSE output may use a Rectified Linear Unit (ReLU) activation function. The MSE output may use a MSE loss function. The MSE loss function may relate to a difference between a reconstructed input signal and the input signal in terms of mean squared error (MSE). Lower MSE relates to better signal reconstruction. To estimate the MSE output two extra dense layers may be used with a ReLU activation function after the flatten layer to have an output of the same size as the input signal.

As outlined above, the class output may contribute to the algorithm learning with a weight of 1. The second output may be weighted using at least one weight. The weight can be varied. The weight can be from 0 to 1. Empirically, it was found that lower weights can lead to slightly higher accuracy. A range of MSE values can be much larger than 1 (which is the maximum class output). Using a weighted second output may allow to balance between the algorithm to learn about the signal reconstruction and about the class output. This may allow increasing the accuracy for both supervised and semi-supervised architectures. For example, for the supervised architecture accuracy may be 91.6% with equal weights (i.e., 1) vs 92.5% with lower MSE weight such as a weight of 0.1. For the semi-supervised architecture accuracy may be 87.7% with equal weights vs 90.6% with lower MSE weight such as a weight of 0.05.

The method may comprise at least one validation step. The validation step may be performed during training of the trainable model. The validation step may be used for monitoring improvement of the training. The validation step may comprise validating the trainable model using at least one validation dataset. The validation dataset, for example, may comprise 1000 non-overlapping manually labelled samples out of the historical biological sensor collected from the 5 healthy volunteers, as described above, wherein the 1000 non-overlapping manual labelled samples used for validation were not used for training.

The method may comprise at least one test step, wherein the test step comprises testing the trained trainable model. The test step may comprise testing the trained trainable model on at least one test dataset. The test step may comprise obtaining performance characteristics of the trained trainable model, e.g. precision, recall, F1-score, area under the curve (AUC).

For example, as test data 1000 non-overlapping manual labelled samples out of the historical biological sensor collected from the 5 healthy volunteers, as described above, were used. The 1000 non-overlapping manual labelled samples used for testing were not used for training. As result, the accuracy was found as follows, wherein a classification threshold of 0.5 was used:
supervised deep learning architecture (with optimizing one loss function): 98.1%
supervised deep learning architecture (with optimizing two loss functions with equal loss functions weight of 1): 98.1%

The classification threshold may denote a quality threshold to label a signal as clean or noisy; ≥0.5 the signal may be classified as clean, <0.5 the signal may be classified as noisy.

For the testing an epoch and regularization strength combination was used that has the highest F1-score, for maintaining a balance between false positives and false negatives. The classification threshold was selected in view that the trained trainable model gives a value between [0, 1], with 0 meaning noisy signal and 1 clean signal. With a classification threshold of 0.5, in case the trained trainable model output value is below 0.5, the signal is regarded as noisy, and clean otherwise. This classification threshold may vary. Techniques for finding the optimal classification threshold are known to the skilled person, e.g. based on ROC curves. For using the semi-supervised model the optimal classification threshold may be used. The method may comprise calculating the optimal classification threshold. Several options for calculating the optimal classification threshold are possible. For example, a sample of the clinical data may be used to estimate the optimal classification threshold and to use that for translating the probabilities (output of the model) into labels rounding on that classification threshold.

Additionally or alternatively, for the testing of the trained trainable model a completely independent labeled dataset may be used. For example, different participants may be used for collecting data for the training set to the ones used for training the model. For example, 1000 non-overlapping samples from the dataset as described in “A quality metric for heart rate variability from photoplethysmogram sensor data”, of M. Zanon et al., PMID: 33018085, DOI: 10.1109/EMBC44109.2020.9175671 may be used as test dataset. As result, the accuracy was found as follows, wherein a classification threshold of 0.5 was used:
supervised deep learning architecture (with optimizing one loss function): 92.5%
supervised deep learning architecture (with optimizing two loss functions with equal loss functions weight of 1): 91.6%
supervised deep learning architecture (with optimizing two loss functions with optimal loss function weight): 92.5%

The following accuracy was found in case of an optimal classification threshold:
supervised deep learning architecture (with optimizing one loss function): 91.8%
supervised deep learning architecture (with optimizing two loss functions with equal loss functions weight of 1): 90.6%
supervised deep learning architecture (with optimizing two loss functions with optimal loss function weight): 92.0%.

It was found that the supervised learning performs better than using a multivariate quality metric, such as proposed in “A quality metric for heart rate variability from photoplethysmogram sensor data”, of M. Zanon et al., PMID: 33018085, DOI: 10.1109/EMBC44109.2020.9175671, because more information for the raw signal is used by the method according to the present invention than the feature based multivariate quality metric. The accuracy of the model using a multivariate quality metric from this paper on the same dataset was 84%. Using the second output relating to the reconstruction was found to help with explainability, i.e. to explain what did the model actually learn during the convolutions from the specific input signal. For example, if the reconstruction can show correct peaks and a sinus waveform, then the model has learned what was important to classify a signal as clean. It was expected that the results might be a bit lower because the model becomes more complicated to learn having two competing loss functions. However, it was found that the method performs better than the multivariate quality metric.

For both supervised and semi-supervised deep learning architectures, using two loss functions may allow to jointly learn to use the labeled signals to classify, and thus, to distinguish clean from noisy signals, and to help the network learn more about the physiology of the signal.

The term “semi-supervised” deep learning architecture as used herein is a broad term and is to be given its ordinary and customary meaning to a person of ordinary skill in the art and is not to be limited to a special or customized meaning. The term specifically may refer, without limitation, to a deep learning architecture learning based on labeled and un-labeled historical biological sensor data. Using unlabeled data in a semi-supervised model may allow improving learning the signal reconstruction and to improve classification performance, especially in cases of new activities/subjects not already included in the original training dataset.

For example, in particular in addition to the labeled PPG dataset described above, as historical biological sensor data for training, an unlabeled PPG dataset may be used. For example, the unlabeled PPG dataset may be set up as follows: Data was collected from 20 healthy volunteers (4 female and 16 male with average age of 32), while performing a series of activities in a supervised manner where the participant would switch activities every 5 minutes. For example, a protocol may be used comprising of multiple activities such as screening and informed consent process (while sitting, at rest), placement of ECG and PPG sensors (while sitting, at rest), baseline (sitting, at rest), paced breathing (ladder of increasing respiratory frequencies from 5 to 20 breaths per minute with steps of 5), 5 minutes of console gameplay (PS4 Aaero), orthostasis (standing, otherwise at rest), mental stress manipulation (Serial 7s [subtraction by 7 from 700, with eyes closed, pronouncing aloud each response]; e.g. as described in Ewing et al 1992), physical activity manipulation (uninterrupted indoor walking along a pre-set circular path; same path for all subjects), baseline (sitting, at rest), retrieve PPG/ECG equipment and debrief. The following table gives a list of an exemplary protocol:

| Activity | Duration |
|---|---|
| Screening & Informed consent process | — |
| (while sitting, at rest) | |
| Placement of ECG and PPG sensors (while | — |
| sitting, at rest) | |
| Baseline (sitting, at rest) | 5 minutes |
| Paced breathing (ladder of increasing | 5 minutes |
| respiratory frequencies from 5 to 20 | |
| breaths per minute with steps of 5) | |
| Console gameplay (PS4 Aaero) | 5 minutes |
| Orthostasis (standing, otherwise | 5 minutes |
| at rest) | |
| Mental stress manipulation (Serial 7s | 5 minutes |
| [subtraction by 7 from 700, with eyes | |
| closed, pronouncing aloud each response]) | |
| Physical activity manipulation (uninterrupted | 5 minutes |
| indoor walking along a pre-set circular | |
| path; same path for all subjects) | |
| Baseline (sitting, at rest) | 5 minutes |
| Retrieve PPG/ECG equipment and debrief | — |

The activities included sitting in a resting position, paced breathing, console gameplay, orthostasis, mental stress manipulation, physical activity, and sitting in a resting position. Some activities are suspected to introduce different level of motion artifacts (e.g., physical activity, orthostasis and console gameplay), while others increase the heart rate and modify the PPG waveform (e.g., paced breathing). For example, in total 37564 non-overlapping PPG signal samples were collected, of 10 seconds length each. For more details of the collection of the unlabeled dataset reference is made to “A quality metric for heart rate variability from photoplethysmogram sensor data”, of M. Zanon et al., PMID: 33018085, DOI: 10.1109/EMBC44109.2020.9175671.

As described above, the training dataset for the semi-supervised model may comprise labeled and unlabeled historical biological sensor data. For example, the manually labeled 9380 balanced signal samples may be used and, additionally, collected unlabeled samples, from the dataset collected in “A quality metric for heart rate variability from photoplethysmogram sensor data”, of M. Zanon et al., PMID: 33018085, DOI: 10.1109/EMBC44109.2020.9175671, may be used.

As described above, the method may comprise the at least one validation step. The validation dataset, for validating trainable model using the semi-supervised deep learning architecture, for example, may comprise 1000 non-overlapping manual labelled samples out of the historical biological sensor collected from the 5 healthy volunteers, as described above, wherein the 1000 non-overlapping manual labelled samples used for validation were not used for training.

As described above, the method may comprise the at least one test step. For example, for testing of the trained trainable model being based on the semi-supervised deep learning architecture as test data 1000 non-overlapping manual labelled samples out of the historical biological sensor collected from the 5 healthy volunteers, as described above, may be used. The 1000 non-overlapping manual labelled samples used for testing were not used for training. The accuracy of the semi-supervised model when using data from the test dataset from the 5 people is:
semi-supervised deep learning architecture (with optimizing two loss functions with equal loss functions weight of 1): 97.9%.

Additionally or alternatively, for the testing, other test data may be used. For example, 1000 non-overlapping samples from the unlabeled dataset collected as described above and in “A quality metric for heart rate variability from photoplethysmogram sensor data”, of M. Zanon et al., PMID: 33018085, DOI: 10.1109/EMBC44109.2020.9175671 may be used as test dataset. The samples used for testing were manually annotated. For example, the dataset used for testing may comprise 796 noisy and 204 clean PPG signals. As result, the accuracy was found as follows, wherein a classification threshold of 0.5 was used:
semi-supervised deep learning architecture (with optimizing two loss functions with equal loss functions weight of 1): 87.7%
semi-supervised deep learning architecture (with optimizing two loss functions with optimal loss function weight): 90.6%.

The following accuracy was found in case of an optimal classification:
semi-supervised deep learning architecture (with optimizing two loss functions with equal loss functions weight of 1): 90.8%
semi-supervised deep learning architecture (with optimizing two loss functions with optimal loss function weight): 91.3%.

The performance of the model may be compared to the performance using a multivariate quality metric, such as proposed in “A quality metric for heart rate variability from photoplethysmogram sensor data”, of M. Zanon et al., PMID: 33018085, DOI: 10.1109/EMBC44109.2020.9175671. The PPG signal was collected simultaneously with ECG signal in order to compare the derived HRV features, and eventually estimate an HRV quality metric per signal, as described in Zanon et al., 2020. The HRV quality metric was computed for each PPG sample signal, and a PPG signal is regarded as trustworthy (i.e., clean) if the HRV quality metric value is below 20. It was found that the semi-supervised learning performs better than using a multivariate quality metric.

The architecture of the semi-supervised deep learning architecture may be identical to the supervised one with the addition of using the unlabeled data in the training step and the extra input parameter zᵢₙₚᵤₜ. Thus, with respect to description of the semi-supervised deep learning architecture reference is made to the supervised deep learning architecture above. For example, the semi-supervised deep learning architecture may comprise five convolutional layers. The first layer may have no dilation, the second one a dilation of 2, and from there on dilation may double for each next layer. In each layer 16 filters may be used. A kernel of size 3, 5, 7 or even other sizes may be used. A regularization strength may be in the range of 0.0005 and 0.002, e.g. 0.0005, 0.001, 0.0015 or 0.002. However, other ranges are possible. The semi-supervised deep learning architecture may comprise a flatten layer after the convolutional layers and before the outputs. The semi-supervised deep learning architecture may comprise an Adam optimizer, e.g. with learning rate of 0.00001 and with a decay where the learning rate is halved every ten or 100 or more epochs. However, other learning rates and learning decay rates are possible. For example, for training, a batch size of 128, and epochs up to 300 may be used. For example, for training, a batch size of 128, and epochs from 50 up to 500 or even more may be used. However, other batch size and epochs are possible. For each epoch the model may be trained once with the labeled data and once with N randomly picked samples from the joined labeled and unlabeled data, where N is 2 times the size of the labeled set.

Manual labeled and unlabeled historical biological sensor data may be used for training the trainable model based on the semi-supervised deep learning architecture. For unlabeled biological sensor data the trainable model may be trained by optimizing the loss function in terms of signal reconstruction and by disregarding the loss function in terms of classification. For training, the same balanced labeled dataset as with the supervised model may be used, and in addition, the unlabeled data described above. An independent dataset may be used for testing the trained trainable model such as the 1000 non-overlapping random samples as described above.

The present invention specifically proposes a novel Wavenet-like dilated convolutional network for cleaning PPG signal data. Obtaining annotated data is costly and time-consuming; however, large amounts of unlabeled data are available. Using a semi-supervised framework based on signal reconstruction allows for learning a good representation of the signal from unlabeled data. It was found that the different approaches to learning control for false positives and false negatives can be performed in different ways, as described herein, while obtaining high overall accuracy. With tuning (specifically an optimal classification threshold), the semi-supervised model can outperform the supervised approach suggesting such that incorporating the large amounts of available unlabeled data can be advantageous.

The present invention proposes a novel approach of classifying data quality of biological sensors by applying a trained trainable model which allows for having signal reconstruction as well as a semi-supervised deep learning model. Signal reconstruction has not been applied before on signals like PPG because it is a technique usually used on images. Using a semi supervised way as proposed by the present invention may require the signal reconstruction technique to combine the information learned by the unlabeled data and the information/class learned by the supervised data. Such an approach was never mentioned before. Semi-supervised approaches have been used before only for other applications but not for biological signals such as a PPG signal quality estimation and just assume the signal is clean.

The method may comprise introducing an extra input parameter zᵢₙₚᵤₜ for training based on the unlabeled dataset. This may allow handling the “missing” labels. The extra input parameter may be a binary value indicating if the specific data is labeled or not. For example, the extra input parameter may be “0” for unlabeled data and “1” for labeled data. The extra input parameter may be multiplied with the class output during the learning process such that the class learning may not be affected by unlabeled data.

For example, the training based on the semi-supervised deep learning architecture may comprise training, firstly, with dataset of labeled data to learn the class label and relevant information for signal reconstruction. The training may, subsequently, comprise training only with a random subset of the unlabeled data to better learn the signal reconstruction. The training using the unlabeled data may further comprise introducing signal physiology that was not included in the labeled training set, e.g., different people, different activities. Annotating data is expensive, and very few annotated datasets are available. However, there is a lot of unlabeled data available. Semi-supervised learning may leverage unlabeled data and makes the most efficient use of small amounts of labeled data.

Using the semi-supervised architecture may allow to expand and/or transfer the proposed model to new dataset. For example, if there is a need to adapt the model to a new scenario, even with less data, such as up to less than 50% of the data used for training the originally trained model, e.g. because there are not enough labeled examples, it is possible to train a reliable model using the semi-supervised approach. It was found that accuracy remains >90% with all algorithms.

The trained trainable model may be trained based on a combination of a supervised and semi-supervised deep learning architecture. The combination may use a combined averaged predictions of the two architectures, taking the average of the probabilities reported by the two architectures into account.

In a further aspect of the present invention, a biological sensor is disclosed. The biological sensor is configured for classifying quality of biological sensor data. The biological sensor comprises at least one measuring unit configured for providing biological sensor data comprising at least one signal. The biological sensor comprises at least one processing unit configured for classifying quality of the signal by using at least one trained trainable model. The trainable model is trained on historical biological sensor data based on a supervised and/or semi-supervised deep learning architecture. The trainable model is trained by optimizing one loss function in terms of classification or two loss functions in terms of signal reconstruction and classification.

Specifically, the biological sensor may be configured for performing the method according to the present invention and/or for being used in the method according to the present invention. For definitions of the features of the biological sensor and for optional features of the biological sensor, reference may be made to one or more of the embodiments of the method as disclosed above or as disclosed in further detail below.

The term “processing unit” as generally used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system and/or, generally, to a device which is configured for performing calculations or logic operations. In particular, the processing unit may be configured for processing basic instructions that drive the computer or system. As an example, the processing unit may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processing unit may be a multi-core processor. Specifically, the processing unit may be or may comprise a central processing unit (CPU). Additionally or alternatively, the processing unit may be or may comprise a microprocessor, thus specifically the processing unit's elements may be contained in one single integrated circuitry (IC) chip. Additionally or alternatively, the processing unit may be or may comprise one or more application-specific integrated circuits (ASICs) and/or one or more field-programmable gate arrays (FPGAs) or the like. The processing unit specifically may be configured, such as by software programming, for performing one or more evaluation operations.

The biological sensor may be a portable photoplethysmogram device. The portable photoplethysmogram device may comprises at least one illumination source and at least one photodetector configured for providing at least one photoplethysmogram. The processing unit may be configured for classifying quality of the photoplethysmogram by using the trained trainable model.

Further disclosed and proposed herein is a computer program including computer-executable instructions for performing the method according to the present invention in one or more of the embodiments enclosed herein when the program is executed on a computer or computer network or a cloud. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps a) and b) and optionally c) as indicated above may be performed by using a computer or a computer network or a cloud, preferably by using a computer program.

Further disclosed and proposed herein is a computer program product having program code means, in order to perform the method according to the present invention in one or more of the embodiments enclosed herein when the program is executed on a computer or computer network or a cloud. Specifically, the program code means may be stored on a computer-readable data carrier and/or on a computer-readable storage medium.

Further disclosed and proposed herein is a data carrier having a data structure stored thereon, which, after loading into a computer or computer network or a cloud, such as into a working memory or main memory of the computer or computer network or a cloud, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network or a cloud. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network or a cloud, for performing the method according to one or more of the embodiments disclosed herein.

The method and devices according to the present invention may provide a number of advantages over known methods and devices of similar kind. Specifically, the present invention may provide an approach to detecting reliable or clean signals from a continuous PPG signal in a real world dataset during everyday life activities. Generally, assessing the quality of PPG signals may be technically challenging as only small amounts of labeled physiological signals and large amounts of unlabeled data are available. By using the method and devices according to the present invention, specifically by using the trainable model based on the semi-supervised deep learning architectures, it may be possible to leverage the large amount of unlabeled data. Thus, it may be possible to interpret the biological sensor data and to ensure that the trainable model learns information about the physiology of the signal by using signal reconstruction during the learning process of the trained model. Moreover, by using the trainable model, it may be possible to reconstruct the signal and at the same time classify the signal as a noisy or clean signal.

US 2019/133468 A1 describes classifying if a person has atrial fibrillation. Thus, the quality obtained by US 2019/133468 A1 is related to a specific disease but not in general for any PPG signal (e.g. as described in FIG. 5 of US 2019/133468 A1). US 2019/133468 A1 describes complex and time consuming preprocessing steps (e.g. in FIG. 4 of US 2019/133468 A1) like identifying movement using a different non-PPG sensor (i.e., IMU), and also removing baseline signal levels. These complex and time consuming preprocessing steps aim to make the quality detection easier. The present invention, in contrast, avoids such complex and time consuming preprocessing steps but can incorporate such steps into the algorithm implicitly in the model. This can allow avoiding these extra steps before the actual model. The model used in US 2019/133468 A1 requires as additional input the motion information, e.g. from additional sensors like accelerometer, that would make any prediction easier (see FIG. 6 of US 2019/133468 A1). For the model according to the present invention, no additional input the motion information is required.

Referring to the computer-implemented aspects of the invention, one or more of the method steps or even all of the method steps of the method according to one or more of the embodiments disclosed herein may be performed by using a computer or computer network or a cloud. Thus, generally, any of the method steps including provision and/or manipulation of data may be performed by using a computer or computer network or a cloud. Generally, these method steps may include any of the method steps, typically except for method steps requiring manual work, such as providing the samples and/or certain aspects of performing the actual measurements.

Specifically, further disclosed herein are:
a computer or computer network or a cloud comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network or a cloud,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network or a cloud, and
a computer program product having program code means, wherein the program code means can be stored or are stored on a storage medium, for performing the method according to one of the embodiments described in this description, if the program code means are executed on a computer or on a computer network or a cloud.

Summarizing and without excluding further possible embodiments, the following embodiments may be envisaged:
Embodiment 1 Computer implemented method for classifying quality of biological sensor data comprising the following steps:
   a) providing biological sensor data obtained by at least one biological sensor, wherein the biological sensor data comprises at least one signal;
   b) classifying quality of the signal by using at least one trained trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.
Embodiment 2 The method according to the preceding embodiment, wherein the biological sensor is at least one portable photoplethysmogram device and the biological sensor data comprises at least one photoplethysmogram obtained by the portable photoplethysmogram device.
Embodiment 3 The method according to the preceding embodiment, wherein the quality is used as quality indicator for heart rate variability data, wherein the quality is used for distinguishing between acceptable and non-acceptable heart rate variability data.
Embodiment 4 The method according to any one of the preceding embodiments, wherein classifying quality comprises discriminating between noisy and clean signals.
Embodiment 5 The method according to any one of the preceding embodiments, wherein the trainable model comprises at least one deep neural network selected from the group consisting of: a Convolutional Neural Network (CNN), a recurrent neural networks (RNN), a Long short-term memory (LSTM).
Embodiment 6 The method according to any one of the preceding embodiments, wherein the method further comprises:
   c) at least one training step, wherein, in the training step, the trainable model is trained on at least one training dataset comprising the historical biological sensor data, based on the supervised and/or semi-supervised deep learning architecture, wherein the trainable model is trained by optimizing the one loss function in terms of classification or the two loss functions in terms of signal reconstruction and classification.
Embodiment 7 The method according to any one of the preceding embodiments, wherein manual labeled historical biological sensor data is used for training the trainable model based on the supervised deep learning architecture.
Embodiment 8 The method according to any one of the preceding embodiments, wherein manual labeled and unlabeled historical biological sensor data is used for training the trainable model based on the semi-supervised deep learning architecture.
Embodiment 9 The method according to the preceding embodiment, wherein for unlabeled biological sensor data the trainable model is trained by optimizing the loss function in terms of signal reconstruction and by disregarding the loss function in terms of classification.
Embodiment 10 The method according to any one of the preceding embodiments, wherein the method comprises at least one pre-processing step comprising one or more of filtering or normalizing the biological sensor data.
Embodiment 11 A biological sensor, wherein the biological sensor is configured for classifying quality of biological sensor data, wherein the biological sensor comprises at least one measuring unit configured for providing biological sensor data comprising at least one signal, wherein the biological sensor comprises at least one processing unit configured for classifying quality of the signal by using at least one trained trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.
Embodiment 12 The biological sensor according to the preceding embodiment, wherein the biological sensor is a portable photoplethysmogram device, wherein the portable photoplethysmogram device comprises at least one illumination source and at least one photodetector configured for providing at least one photoplethysmogram, wherein the processing unit is configured for classifying quality of the photoplethysmogram by using the trained trainable model.
Embodiment 13 The biological sensor according to any one of the two preceding embodiments, wherein the biological sensor is configured for performing the method according to any one of the preceding embodiments referring to a method.
Embodiment 14 A computer program comprising instructions which, when the program is executed by a biological sensor according to any one of the preceding embodiments referring to a biological sensor, cause the biological sensor to carry out steps a) to b) and optionally step c) of the method according to any one of the preceding embodiments referring to a method.
Embodiment 15 A computer-readable storage medium comprising instructions which, when executed by a biological sensor according to any one of the preceding embodiments referring to a biological sensor, cause the biological sensor to carry out steps a) to b) and optionally step c) of the method according to any one of the preceding embodiments referring to a method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows a flow diagram of a computer implemented method for classifying quality of biological sensor data and an embodiment of a biological sensor in a schematic view;

FIGS. 2A to 2D show exemplary biological sensor data comprising clean (FIGS. 2A and 2B) and noisy signals (FIGS. 2C and 2D);

FIGS. 3A to 3C show embodiments of a supervised deep learning architecture in a schematic view;

FIGS. 4A and 4B show exemplary reconstructed signals for a supervised deep learning architecture;

FIGS. 5A and 5B show embodiments of a semi-supervised deep learning architecture in a schematic view;

FIGS. 6A and 6B show an exemplary reconstructed signal for a supervised and a semi-supervised deep learning architecture;

FIG. 7 shows a histogram of activities of randomly selected PPG samples;

FIGS. 8A to 8C show performance data of different deep learning architectures for a first number of labeled data;

FIGS. 9A to 9C show performance data of different deep learning architectures for a second number of labeled data; and

FIG. 10 shows performance data of a semi-supervised deep learning architecture for different shares of labeled signals.

### DETAILED DESCRIPTION OF THE EMBODIMENTS

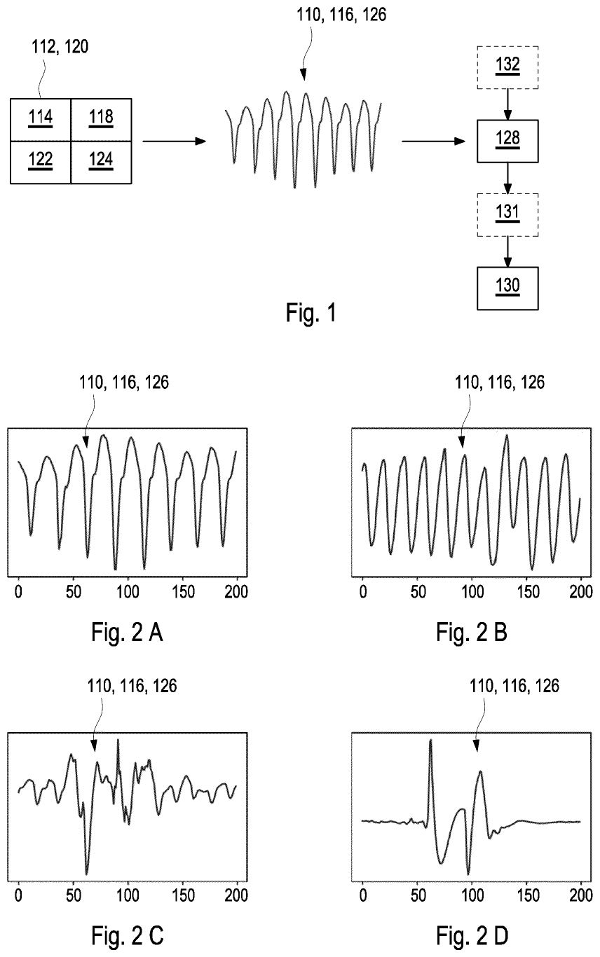

FIG. 1 shows a flow diagram of a computer implemented method for classifying quality of biological sensor data 110 and an exemplary embodiment of a biological sensor 112 in a schematic view. The biological sensor 112 is configured for classifying quality of biological sensor data 110. The biological sensor 112 comprises at least one measuring unit 114 configured for providing biological sensor data 110 comprising at least one signal 116. The biological sensor 112 comprises at least one processing unit 118 configured for classifying quality of the signal 116 by using at least one trained trainable model 119 (not shown in FIG. 1). The trainable model 119 is trained on historical biological sensor data based on a supervised 134 and/or semi-supervised deep learning architecture 188 (not shown in FIG. 1). The trainable model 119 is trained by optimizing one loss function in terms of classification or two loss functions in terms of signal reconstruction and classification.

The biological sensor 112 may be a portable photoplethysmogram device 120. The portable photoplethysmogram device 120 may comprises at least one illumination source 122 and at least one photodetector 124 configured for providing at least one photoplethysmogram 126. The processing unit 118 may be configured for classifying quality of the photoplethysmogram 126 by using the trained trainable model 119.

The biological sensor 112 may specifically be configured for performing the method for classifying quality of biological sensor data 110 and/or for being used in the method for classifying quality of biological sensor data 110. An exemplary embodiment of the method for classifying quality of biological sensor data 110 is shown in the flow diagram of FIG. 1.

The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlap-ping fashion. The method may comprise further method steps which are not listed.

The method comprises the following steps:
a) (denoted by reference number 128) providing biological sensor data 110 obtained by the at least one biological sensor 112, wherein the biological sensor data 110 comprises the at least one signal 116;
b) (denoted by reference number 130) classifying quality of the signal 116 by using at least one trained trainable model 119, wherein the trainable model 119 is trained on historical biological sensor data based on a supervised 134 and/or semi-supervised deep learning architecture 188, wherein the trainable model 119 is trained by optimizing one loss function in terms of classification or two loss functions in terms of signal reconstruction and classification.

As outlined above, the biological sensor 112 may be the at least one portable photoplethysmogram device 120. The biological sensor data 110 may comprise the at least one photoplethysmogram 126 obtained by the portable photoplethysmogram device 120. As an example, the quality may be used as quality indicator for heart rate variability data. The quality may be used for distinguishing between acceptable and non-acceptable heart rate variability data.

Further, either raw signals may be used, or processed or preprocessed signals may be used, thereby generating secondary signals, which may also be used as sensor signals 116. The method may comprise at least one pre-processing step (denoted by reference number 131) comprising one or more of filtering or normalizing the biological sensor data 110. As shown in FIG. 1, the pre-processing step may specifically be performed in between step a) and b). For example, in case of a signal 116 of the PPG device 120, a bandpass filter may be used. Additionally, the signal 116 may be normalized so that the values are around 0. However, preprocessing can be different for different signals 116 depending on the physiology.

In the method, classifying quality may comprise discriminating between noisy and clean signals. Exemplary biological sensor data 110 are shown in FIGS. 2A to 2D. Therein, specifically, signals 116 as exemplarily comprised by the biological sensor data 110 are shown. In the examples of FIGS. 2A to 2D, the biological sensor data 110 comprises data from the photoplethysmogram 126. Thus, in this example, the signal 116 comprised by the biological sensor data 110 may be a 10 second interval of a PPG signal with 20 Hz sampling frequency resulting in 200 PPG data points. Clean signals 116 are shown in FIGS. 2A and 2B and noisy signals 116 are shown in FIGS. 2C and 2D.

Turning back to FIG. 1. The method may further comprise, specifically prior to step a):
c) (denoted by reference number 132) at least one training step, wherein, in the training step, the trainable model 119 is trained on at least one training dataset comprising the historical biological sensor data, based on the supervised 134 and/or semi-supervised deep learning architecture 188, wherein the trainable model 119 is trained by optimizing the one loss function in terms of classification or the two loss functions in terms of signal reconstruction and classification.

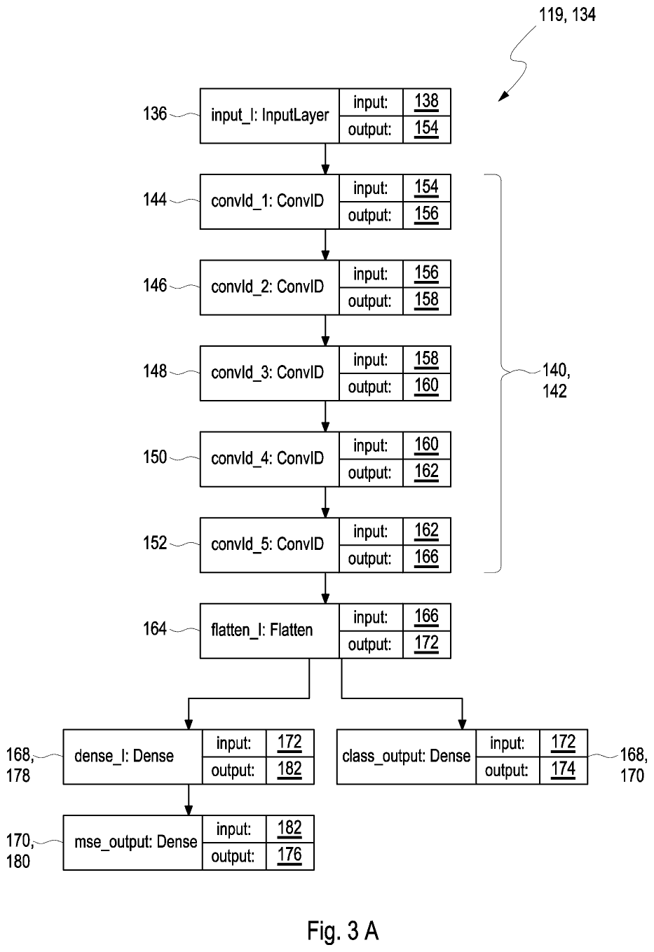

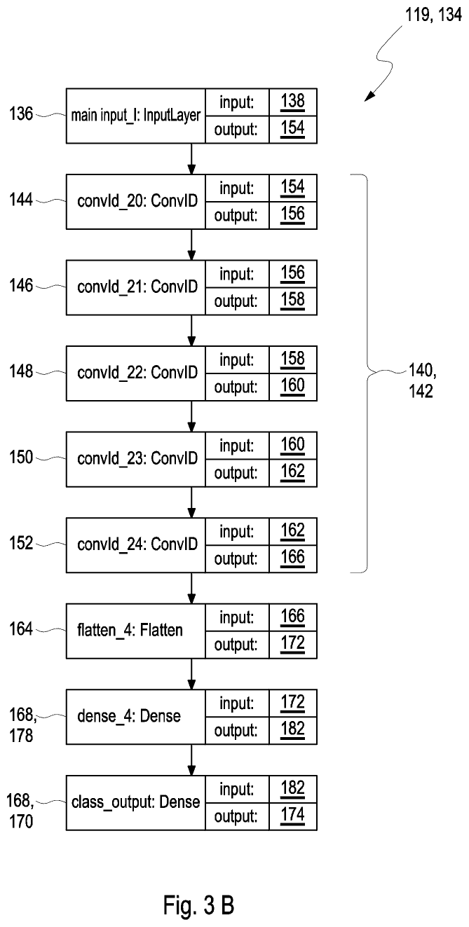

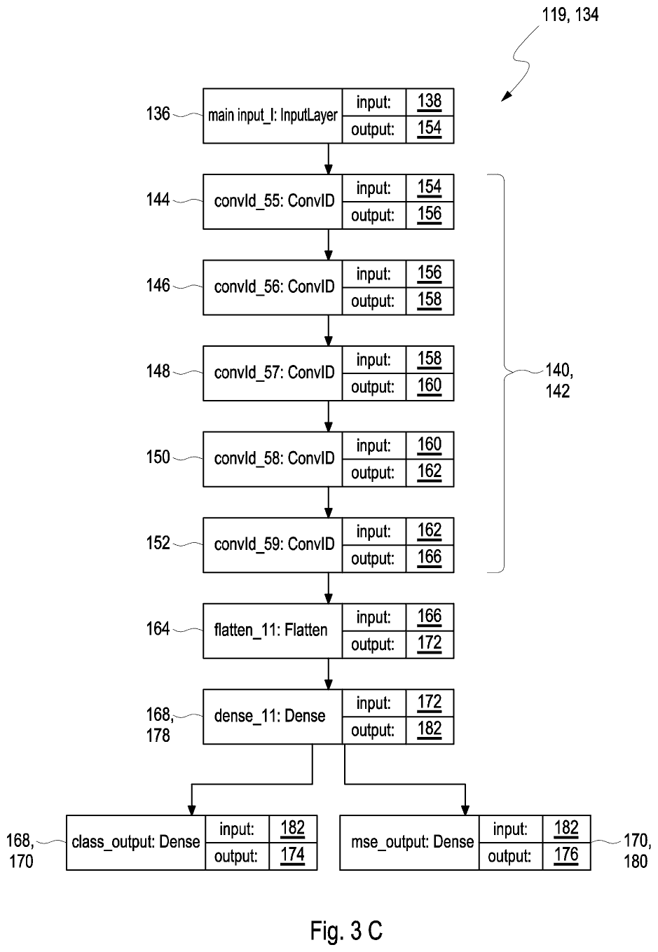

The trainable model 119 is trained on historical biological sensor data based on a supervised 134 and/or semi-supervised deep learning architecture 188. Exemplary embodiments of a supervised deep learning architecture 134 are shown in FIGS. 3A to 3C in a schematic view.

The supervised deep learning architecture 134 may comprise at least one input layer 136 receiving the historical biosensor data and/or preprocessed historical biosensor data. For example, as input (denoted by reference number 138) filtered PPG signals of 10 seconds each with 20 Hz frequency may be used. Thus, the input 138 may comprise a signal 116 comprising 200 values, such as signals 116 exemplarily described in FIG. 2.

In the method, manual labeled historical biological sensor data may be used for training the trainable model 119 based on the supervised deep learning architecture 134. As historical biological sensor data, a manual labeled PPG dataset may be used. For example, a training dataset of biological sensor data 110 may be set up as follows: Data was collected from 5 healthy volunteers (1 female and 4 male with average age of 33) without any supervision, during their normal daily activities, or during their night sleep. In total 13547 non-overlapping PPG signal samples were collected, of 10 seconds length each. The signals may be manually labeled by experts according to the instructions of Elgendi, M., “Optimal signal quality index for photoplethysmogram signals”, Scientific Reports, 3(4), 2016. 8305 noisy, and 5242 clean PPG signals were categorized. For example, a balanced dataset of 9380 labeled signal samples may be used as training dataset, specifically for training.

The training step may comprise preprocessing the historical biological sensor data, e.g. filtering the PPG signals of 10 seconds each with 20 Hz frequency, with in total 200 data points. The labels may be provided for each input signal for training with “0” indicating a noisy and “1” a clean signal.

The supervised deep learning architecture 134 may comprise a plurality of convolutional layers 140, in particular a stack of convolutional layers 142. The convolutional layers may be designed with dilation. The convolutional layers may be configured for dilated convolution. The supervised deep learning architecture 134 may comprise a WaveNet neural network, as described in further detail above. However, other deep neural networks, such as recurrent neural networks (RNNs) and/or a Long short-term memory (LSTMs) are also feasible. The supervised deep learning architecture 134 may comprise causal padding in each convolutional layer.

In the exemplary embodiments shown in FIGS. 3A to 3C, the supervised deep learning architecture 134 may comprise five convolutional layers 144, 146, 148, 150, 152. The first layer 144 may have no dilation, the second one 146 a dilation of 2, and from there on dilation may double for each next layer. In each layer, 16 filters may be used.

A kernel of size 3, 5, 7 or even other sizes may be used. A regularization strength may be in the range of 0.0005 and 0.002, e.g. 0.0005, 0.001, 0.0015 or 0.002. However, other ranges are possible. As can be seen in FIGS. 3A to 3C, output of a preceding layer may form input of a following layer. For example, output of the input layer 136 (denoted by reference number 154) may form input of the first convolutional layer 144, output of the first convolutional layer 144 (denoted by reference number 156) may form input of the second convolutional layer 146, output of the second convolutional layer 146 (denoted by reference number 158) may form input of the third convolutional layer 148, output of the third convolutional layer 148 (denoted by reference number 160) may form input of the fourth convolutional layer 150 and output of the fourth convolutional layer 150 (denoted by reference number 162) may form input of the fifth convolutional layer 152.

The supervised deep learning architecture 134 may comprise an Adam optimizer, e.g. with learning rate of 0.00001 and with a decay where the learning rate is halved every ten or 100 or more epochs. However, other learning rates are possible. For example, for training, a batch size of 128, and epochs up to 300 may be used. For example, for training, a batch size of 128, and epochs from 50 up to 500 or even more may be used. However, other batch size and epochs are possible.

As shown in FIGS. 3A to 3C, the supervised deep learning architecture 134 may comprise a flatten layer 164 after the convolutional layers and before the outputs. The flatten layer 164 may be designed to transform a matrix output of the convolutional layers (denoted as reference number 166) into a dense layer 168. Thus, in this example, the transformed matrix output of the convolutional layers as output of the flatten layer 164 (denoted by reference number 172) may form input of the dense layer 168.

The deep learning architecture, specifically the supervised deep learning architecture 134, may comprise as final layer, in particular the dense layer 168, an output layer 170 comprising one or two paths. The exemplary embodiments shown in FIGS. 3A and 3C show the supervised deep learning architecture 134 with the output layer 170 comprising two paths. In this example, the supervised deep learning architecture 134 may be trained by optimizing two loss functions in terms of signal reconstruction and classification. Alternatively, however, the output layer 170 may also comprise only one path as exemplarily shown in FIG. 3B. In this example, the supervised deep learning architecture may be trained by optimizing one loss function in terms of classification. In the examples of FIGS. 3A and 3C, each of the paths may comprise an output. Specifically, the supervised deep learning architecture 134 may comprise two outputs, a first (denoted by reference number 174) and a second output (denoted by reference number 176).

The first output 174 may be a class output providing the classified quality. The class output may use the first loss function. The first loss function may relate to classification loss. In the exemplary embodiment of FIG. 3A, the class output may take the flatten layer's output 172 as input. Alternatively, as exemplarily shown in FIGS. 3B and 3C, the class output may use the dense layer's output 182 as input, in particular instead of the flatten layer's output 172. The class output may use a sigmoid activation function. The class output may use a binary crossentropy loss function to provide a probability between 0 and 1, with a value over 0.5 indicating that the signal 116 is clean.

The second output 176 may be a mean squared error (MSE) output providing a measure for a difference between the input 138 and reconstructed signal 184 after the convolutions. The MSE output may use a Rectified Linear Unit (ReLU) activation function. The MSE output may use a MSE loss function. The MSE loss function may relate to a difference between a reconstructed input signal and the input signal 138 in terms of mean squared error (MSE). Lower MSE relates to better signal reconstruction. To estimate the MSE output two extra dense layers, i.e. a first extra dense layer 178 and a second extra dense layer 180 as shown in FIGS. 3A and 3C, may be used with a ReLU activation function after the flatten layer 164 to have an output of the same size as the input signal 138. An output of the first extra dense layer 178 (denoted by reference number 182) may form input for the second extra dense layer 180.

The method may comprise at least one validation step. The validation step may be performed during training of the trainable model 119. The validation step may be used for monitoring improvement of the training. The validation step may comprise validating the trainable model 119 using at least one validation dataset. The validation dataset, for example, may comprise 1000 non-overlapping manual labelled samples out of the historical biological sensor collected from the 5 healthy volunteers, as described above, wherein the 1000 non-overlapping manual labelled samples used for validation were not used for training.

The method may comprise at least one test step, wherein the test step comprises testing the trained trainable model 119. The test step may comprise testing the trained trainable model 119 on at least one test dataset. The test step may comprise obtaining performance characteristics of the trained trainable model 119, e.g. precision, recall, F1-score, area under the curve (AUC).

For example, as test data 1000 non-overlapping manual labelled samples out of the historical biological sensor collected from the 5 healthy volunteers, as described above, were used. The 1000 non-overlapping manual labelled samples used for testing were not used for training. As result, the accuracy was found as follows, wherein a classification threshold of 0.5 was used:
supervised deep learning architecture 134 (with optimizing one loss function as exemplarily shown in FIG. 3B): 98.1%
supervised deep learning architecture 134 (with optimizing two loss functions with equal loss functions weight of 1, as exemplarily shown in FIG. 3C): 98.1%

For the testing an epoch and regularization strength combination was used that has the highest F1-score, for maintaining a balance between false positives and false negatives. The classification threshold was selected in view that the trained trainable model 119 gives a value between [0, 1], with 0 meaning noisy signal and 1 clean signal. With a classification threshold of 0.5, in case the trained trainable model 119 output value is below 0.5, the signal 116 is regarded as noisy, and clean otherwise. This classification threshold may vary. Techniques for finding the optimal classification threshold are known to the skilled person, e.g. based on ROC curves.

Additionally or alternatively, for the testing of the trained trainable model 119, a completely independent labeled dataset may be used. For example, different participants may be used for collecting data for the training set to the ones used for training the model 119. For example, 1000 non-overlapping samples from the dataset as described in “A quality metric for heart rate variability from photoplethysmogram sensor data”, of M. Zanon et al., PMID: 33018085, DOI: 10.1109/EMBC44109.2020.9175671 may be used as test dataset. As result, the accuracy was found as follows, wherein a classification threshold of 0.5 was used:
supervised deep learning architecture 134 (with optimizing one loss function, as exemplarily shown in FIG. 3B): 92.5%
supervised deep learning architecture 134 (with optimizing two loss functions with equal loss functions weight of 1, as exemplarily shown in FIG. 3C: 91.6%
supervised deep learning architecture 134 (with optimizing two loss functions with optimal loss function weight, as exemplarily shown in FIG. 3C: 92.5%

The following accuracy was found in case of an optimal classification threshold:
supervised deep learning architecture 134 (with optimizing one loss function, as exemplarily shown in FIG. 3B): 91.8%
supervised deep learning architecture 134 (with optimizing two loss functions with equal loss functions weight of 1, as exemplarily shown in FIG. 3C: 90.6%
supervised deep learning architecture 134 (with optimizing two loss functions with optimal loss function weight, as exemplarily shown in FIG. 3C: 92.0%.

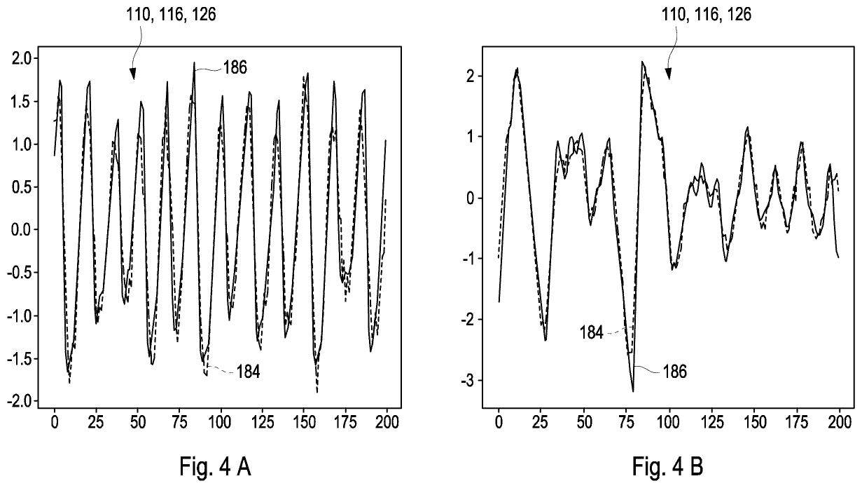

In the FIGS. 4A and 4B, the dataset described in “A quality metric for heart rate variability from photoplethysmogram sensor data”, of M. Zanon et al., PMID: 33018085, DOI: 10.1109/EMBC44109.2020.9175671 was used. In FIGS. 4A and 4B, exemplary reconstructed signals 184 for the supervised deep learning architecture 134 are shown. The supervised deep learning architecture 134 may specifically be embodied according to any one of embodiments shown in FIGS. 3A to 3C. However, other embodiments are also feasible. In FIGS. 4A and 4B, reconstructed signals 184 are shown together with original signals 186. The two example signals 116 shown in FIGS. 4A and 4B were identified, based on their HRV quality metric value, as clean signals since their HRV multivariate quality metric is below 20, even though the signal 116 shown in FIG. 4B is clearly a noisy signal. The HRV quality metric value, also referred to as the HRV multivariate quality metric, may be a multivariate quality metric as proposed in “A quality metric for heart rate variability from photoplethysmogram sensor data”, of M. Zanon et al., PMID: 33018085, DOI: 10.1109/EMBC44109.2020.9175671. As can be seen in FIGS. 4A and 4B, the supervised deep learning architecture 134 may be able to accurately reconstruct the peaks of the clean (FIG. 4A) and noisy (FIG. 4B) signal. The supervised deep learning architecture 134 may correctly classify the signal 116 in FIG. 4A as clean (classification=1) and the signal 116 in FIG. 4B as noisy (classification=0) contrarily to the classification using the HRV quality metric value that incorrectly labeled both signals 116 as clean.

The exemplary reconstructed signals 184 in FIGS. 4A and 4B are shown using the class output and the MSE output equally weighted, for example with an equal weight of 1. The classification threshold may be 0.5. For FIGS. 4A and 4B 300 epochs and regularization strength of 0.002 were used. In FIG. 4A, a signal, which was manually labeled as clean (0 meaning noisy signal and 1 clean signal) is shown, wherein the prediction of the supervised deep learning architecture gives 1 (exact prediction of algorithm 0.989). In comparison, the HRV quality metric gives 7.5, wherein for the HRV quality metric a PPG signal is regarded as clean if the HRV quality metric value is below 20. In FIG. 4B, a signal which was manually labelled as noisy, is shown, wherein the prediction of the supervised deep learning architecture gives 0 (exact prediction of algorithm 0.002). In comparison the HRV quality metric gives 9.61. The signal 116 shown in FIG. 4A was found to be correctly predicted as clean with both models, the supervised deep learning architecture 134 and the HRV multivariate quality metric. The reconstructed signal 184 may match the original signals 186 very well and may identify the peaks of the original signals 186 correctly. As can be seen in FIG. 4B, the supervised deep learning architecture 134 correctly predicts the signal 116 to be noisy. The HRV multivariate quality metric, however, of 9.61 would suggest the signal 116 to be clean (9.61<20). Additionally, it was found that the signal reconstruction with the MSE output contributing with a smaller weight, such as a weight lower than 1, for example a weight of 0.1, changes in that the amplitude of the reconstructed signal 194 is diminished but the peaks of the original signal 186 and the reconstructed signal 184 still match like in the case with equal weights.

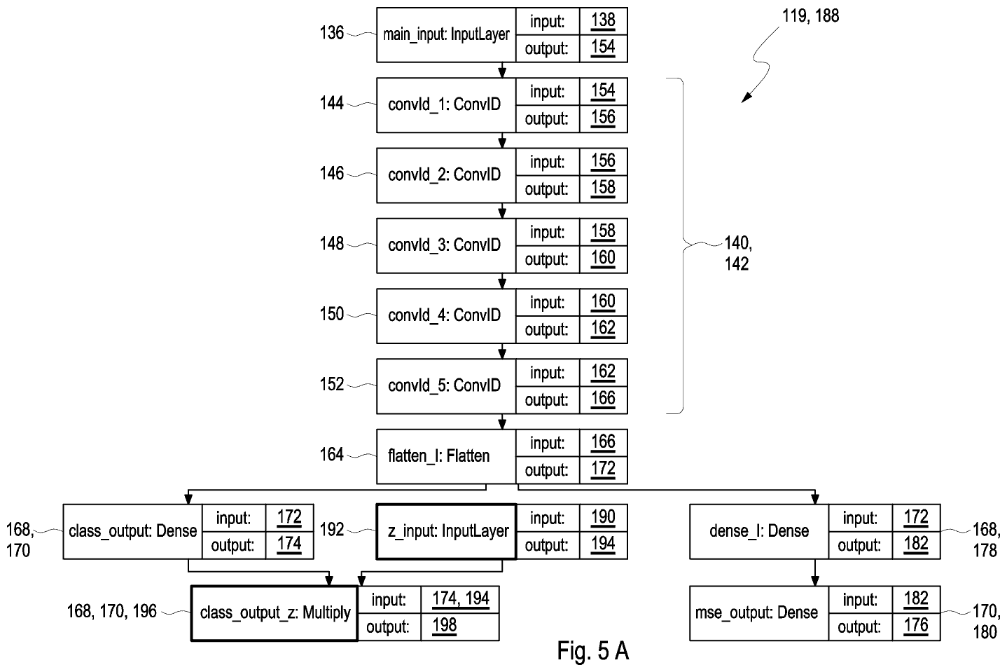

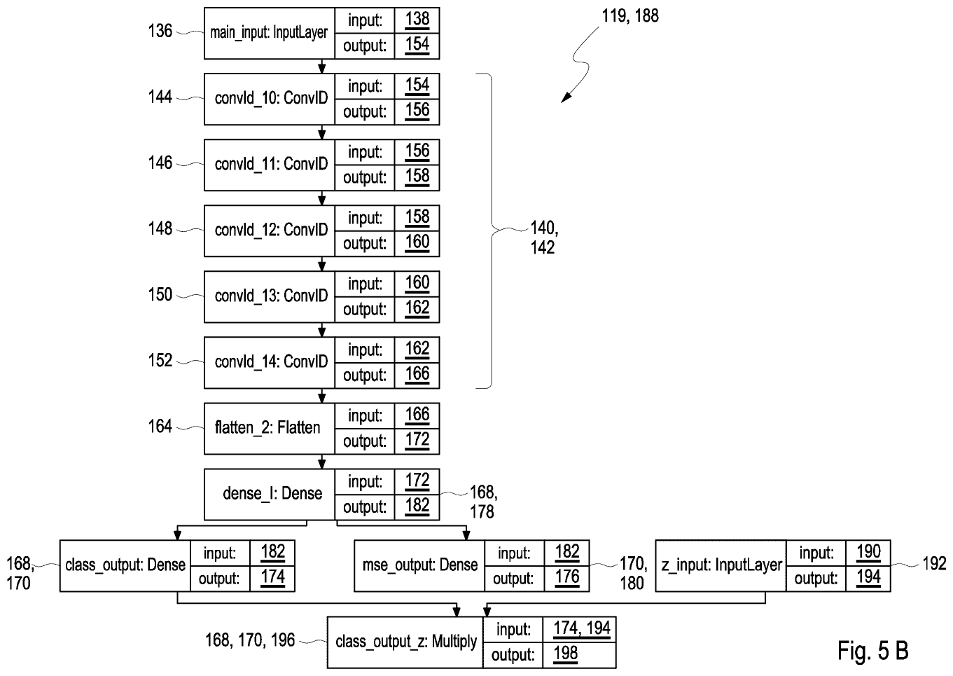

FIGS. 5A and 5B show exemplary embodiments of a semi-supervised deep learning architecture 188 in a schematic view. The semi-supervised deep learning architecture 188 may widely correspond to the supervised deep learning architecture 134 as shown in FIGS. 3A to 3C. Thus, for the description of the semi-supervised deep learning architecture 188, reference is made to the description of FIGS. 3A to 3C.

In the example of FIGS. 5A and 5B, the semi-supervised deep learning architecture may comprise the five convolutional layers 144, 146, 148, 150, 152. The first layer 144 may have no dilation, the second 146 one a dilation of 2, and from there on dilation may double for each next layer. In each layer 16 filters may be used. A kernel of size 3, 5, 7 or even other sizes may be used. A regularization strength may be in the range of 0.0005 and 0.002, e.g. 0.0005, 0.001, 0.0015 or 0.002. However, other ranges are possible. The semi-supervised deep learning architecture 188 may comprise the flatten layer 164 after the convolutional layers 144, 146, 148, 150, 152 and before the outputs. The semi-supervised deep learning architecture 188 may comprise an Adam optimizer, e.g. with learning rate of 0.00001 and with a decay where the learning rate is halved every ten or 100 or more epochs. However, other learning rates are possible. For example, for training, a batch size of 128, and epochs up to 300 may be used. For example, for training, a batch size of 128, and epochs from 50 up to 500 or even more may be used. However, other batch size and epochs are possible. For each epoch, the model 119 may be trained once with the labeled data and once with N randomly picked samples from the joined labeled and unlabeled data, where N is 2 times the size of the labeled set.

In the example of the semi-supervised deep learning architecture 188, the method may comprise introducing an extra input parameter zᵢₙₚᵤₜ (denoted by reference number 190) for training based on an unlabeled dataset. This may allow handling the “missing” labels. The extra input parameter 190 may be a binary value indicating if the specific data is labeled or not. For example, the extra input parameter 190 may be “0” for unlabeled data and “1” for labeled data. The extra input parameter 190 may be multiplied with the class output during the learning process such that the class learning may not be affected by unlabeled data.

Thus, as can be seen in FIGS. 5A and 5B, the semi-supervised deep learning architecture 188 may comprise an additional input layer 192. The additional input layer 192 may be configured for assigning a value of “0” for unlabeled data and “1” for labeled data to the extra input parameter zᵢₙₚᵤₜ 190. An output of the additional input layer 192 (denoted by reference number 194) may form input for an additional output layer 196. In the additional output layer 196, the first output 174, specifically the class output, and the extra input parameter 190 comprised by the output 194 may be multiplied to obtain resulting output 198. In the exemplary embodiment of FIG. 5A, the class output may take the flatten layer's output 172 as input. Alternatively, as exemplarily shown in FIG. 5B, the class output may use the dense layer's output 182 as input, in particular instead of the flatten layer's output 172.

Manual labeled and unlabeled historical biological sensor data may be used for training the trainable model 119 based on the semi-supervised deep learning architecture 188. For unlabeled biological sensor data, the trainable model 119 may be trained by optimizing the loss function in terms of signal reconstruction and by disregarding the loss function in terms of classification. For training, the same balanced labeled dataset as with the supervised model 134 may be used, and in addition, unlabeled data described in the following:

For example, in particular in addition to the labeled PPG dataset described above, as historical biological sensor data for training, an unlabeled PPG dataset may be used. For example, the unlabeled PPG dataset may be set up as follows: Data was collected from 20 healthy volunteers (4 female and 16 male with average age of 32), while performing a series of activities in a supervised manner where the participant would switch activities every 5 minutes. For example, a protocol may be used comprising of multiple activities such as screening and informed consent process (while sitting, at rest), placement of ECG and PPG sensors (while sitting, at rest), baseline (sitting, at rest), paced breathing (ladder of increasing respiratory frequencies from 5 to 20 breaths per minute with steps of 5), 5 minutes of console gameplay (PS4 Aaero), orthostasis (standing, otherwise at rest), mental stress manipulation (Serial 7s [subtraction by 7 from 700, with eyes closed, pronouncing aloud each response]; e.g. as described in Ewing et al 1992), physical activity manipulation (uninterrupted indoor walking along a pre-set circular path; same path for all subjects), baseline (sitting, at rest), retrieve PPG/ECG equipment and debrief.

The activities included sitting in a resting position, paced breathing, console gameplay, orthostasis, mental stress manipulation, physical activity, and sitting in a resting position. Some activities are suspected to introduce different level of motion artifacts (e.g., physical activity, orthostasis and console gameplay), while others increase the heart rate and modify the PPG waveform (e.g., paced breathing). For example, in total 37564 non-overlapping PPG signal samples were collected, of 10 seconds length each. For more details of the collection of the unlabeled dataset reference is made to “A quality metric for heart rate variability from photoplethysmogram sensor data”, of M. Zanon et al., PMID: 33018085, DOI: 10.1109/EMBC44109.2020.9175671.

As described above, the training dataset for the semi-supervised model 188 may comprise labeled and unlabeled historical biological sensor data. For example, the labeled 9380 balanced signal samples may be used and, additionally, collected unlabeled samples may be used.

As described above, the method may comprise the at least one validation step. The valida-tion dataset, for validating trainable model using the semi-supervised deep learning archi-tecture 188, for example, may comprise 1000 non-overlapping manual labelled samples out of the historical biological sensor collected from the 5 healthy volunteers, as described above, wherein the 1000 non-overlapping manual labelled samples used for validation were not used for training.

As described above, the method may comprise the at least one test step. For example, for testing of the trained trainable model 119 being based on the semi-supervised deep learning architecture 188 as test data 1000 non-overlapping manual labelled samples out of the historical biological sensor collected from the 5 healthy volunteers, as described above, may be used. The 1000 non-overlapping manual labelled samples used for testing were not used for training. The accuracy of the semi-supervised model when using data from the test dataset from the 5 people is:
semi-supervised deep learning architecture (with optimizing two loss functions with equal loss functions weight of 1): 97.9%.

Additionally or alternatively, for the testing, other test data may be used. For example, 1000 non-overlapping samples from the unlabeled dataset collected as described above and in “A quality metric for heart rate variability from photoplethysmogram sensor data”, of M. Zanon et al., PMID: 33018085, DOI: 10.1109/EMBC44109.2020.9175671 may be used as test dataset. The samples used for testing were manually annotated. For example, the dataset used for testing may comprise 796 noisy and 204 clean PPG signals. As result, the accuracy was found as follows, wherein a classification threshold of 0.5 was used:
semi-supervised deep learning architecture 188 (with optimizing two loss functions with equal loss functions weight of 1): 87.7%
semi-supervised deep learning architecture 188 (with optimizing two loss functions with optimal loss function weight): 90.6%.

The following accuracy was found in case of an optimal classification:
semi-supervised deep learning architecture 188 (with optimizing two loss functions with equal loss functions weight of 1): 90.8%
semi-supervised deep learning architecture 188 (with optimizing two loss functions with optimal loss function weight): 91.3%.

The performance of the model 119 may be compared to the performance using a multivariate quality metric, such as proposed in “A quality metric for heart rate variability from photoplethysmogram sensor data”, of M. Zanon et al., PMID: 33018085, DOI: 10.1109/EMBC44109.2020.9175671. The PPG signal was collected simultaneously with ECG signal in order to compare the derived HRV features, and eventually estimate an HRV quality metric per signal, as described in Zanon et al., 2020. The HRV quality metric was computed for each PPG sample signal, and a PPG signal is regarded as trustworthy (i.e., clean) if the HRV quality metric value is below 20. It was found that the semi-supervised learning performs better than using a multivariate quality metric.

The training based on the semi-supervised deep learning architecture 188 may comprise training, firstly, with dataset of labeled data to learn the class label and relevant information for signal reconstruction. The training may, subsequently, comprise training only with a random subset of the unlabeled data to better learn the signal reconstruction. The training using the unlabeled data may further comprise introducing signal physiology that was not included in the labeled training set, e.g., different people, different activities. Annotating data is expensive, and very few annotated datasets are available. However, there is a lot of unlabeled data available. Semi-supervised learning may leverage unlabeled data and makes the most efficient use of small amounts of labeled data.

For both supervised 134 and semi-supervised deep learning architectures 188, the use of two loss functions may allow to jointly learn to use the labeled signals to classify, and thus, to distinguish clean from noisy signals, and to help the network learn more about the physiology of the signal 116.

Further, in the method, the trained trainable model 119 may be trained based on a combination 216 (not shown in FIG. 5) of a supervised 134 and semi-supervised deep learning architecture 188. The combination 216 may use a combined averaged predictions of the two architectures, taking the average of the probabilities reported by the two architectures into account.

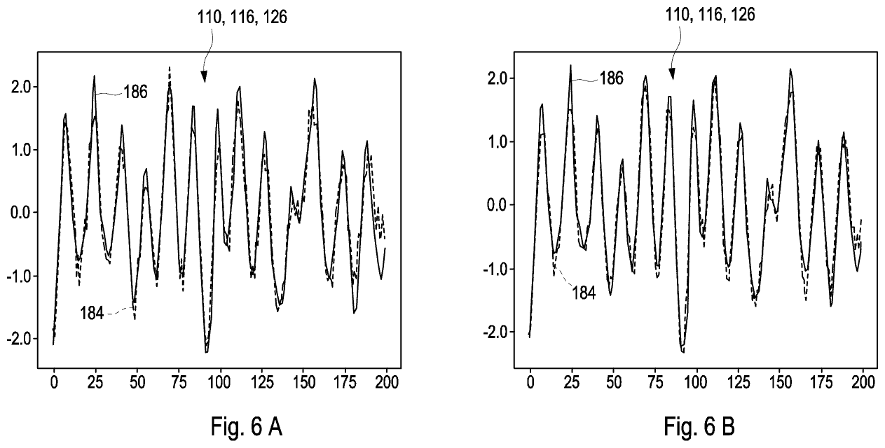

In the FIGS. 6A and 6B, the dataset described in “A quality metric for heart rate variability from photoplethysmogram sensor data”, of M. Zanon et al., PMID: 33018085, DOI: 10.1109/EMBC44109.2020.9175671 was used. In FIGS. 6A and 6B, an exemplary reconstructed signal 184 for a supervised 134 (FIG. 6A) and a semi-supervised deep learning architecture 188 (FIG. 6B) are shown. Specifically, the reconstruction shown in FIGS. 6A and 6B is for the same original signal 186 using the supervised 134 and the semi-supervised deep learning architecture 188. The supervised 134 and the semi-supervised deep learning architecture 188 may be embodied according to any embodiment shown in FIGS. 3A to 3C and 5A and 5B, respectively. In both Figures, the reconstructed signal 184 is shown together with the original signal 186.

When comparing the signal reconstruction of the semi-supervised 188 (FIG. 6B) to the supervised deep learning architecture 134 (FIG. 6A), it can be seen that the former may improve the signal reconstruction of certain signal samples like the one depicted, and also may improve the accuracy of the prediction. The classification threshold may be 0.5. In FIGS. 6A and 6B, a signal manually labeled as clean is shown (0 meaning noisy signal and 1 clean signal). In FIG. 6A, the prediction of the supervised deep learning architecture is shown which gives 0 (exact prediction of algorithm 0.197). 300 epochs and regularization strength of 0.002 were used. In FIG. 6B, the prediction of the semi-supervised deep learning architecture is shown which gives 1 (exact prediction of algorithm 0.649). The semi-supervised model has learned the physiology of the signal better due to the unlabeled data used during the training. Therefore, the peaks of the signal are much clearer estimated which results in a better prediction. 400 epochs and regularization strength of 0.0005 were used. In comparison, the HRV quality metric gives 14.81, wherein for the HRV quality metric a PPG signal is regarded as clean if the HRV quality metric value is below 20. In the example shown in FIGS. 6A and 6B, the supervised deep learning architecture 134 incorrectly classifies the signal 116 as noisy, whereas the semi-supervised deep learning architecture 188 correctly classifies the signal 116 as clean. This shows that, generally, the semi-supervised deep learning architecture 188 may be more accurate in terms of signal reconstruction than the supervised deep learning architecture 134 resulting in higher true positive rates.

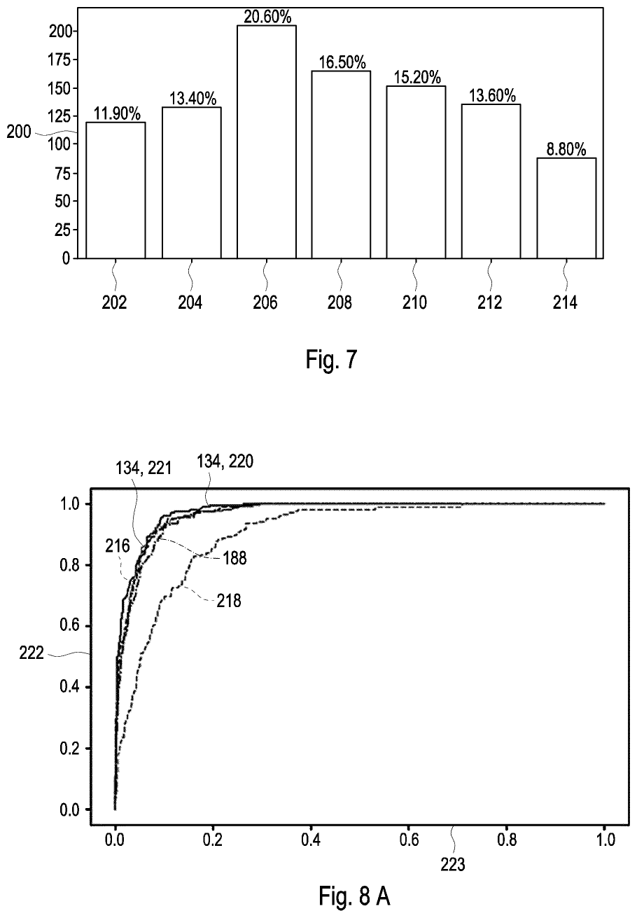

As outlined above, the method as described with respect to FIG. 1 may further comprise the at least one test step, wherein for testing of the trained trainable model 119, the at least one test dataset may be used. An amount of data points 200 in the test dataset for each activity of the experiment protocol is shown in FIG. 7. Therein, the amount of data points for rest begin 202, for breathing 204, for gaming 206, for orthostasis 208, for mental stress 210, for physical activity 212 and for rest end 214 is shown in total numbers and relative to the total amount of samples.

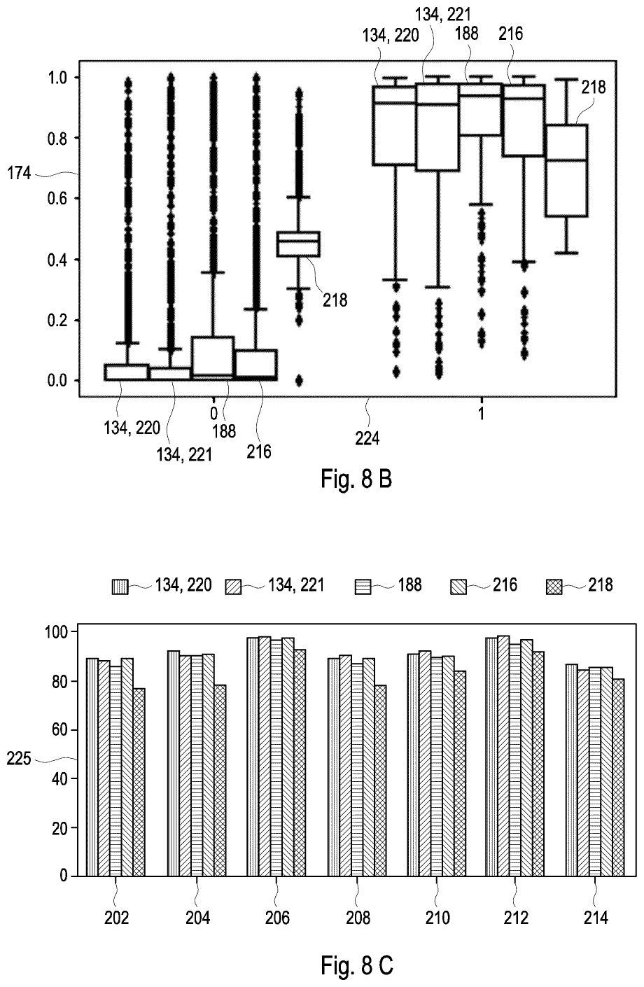

FIGS. 8A to 8C show performance data of different deep learning architectures for a first number of labeled data used for training. The first number of labeled data may comprise 100% of the balanced dataset of 9380 labeled signal samples. For the semi-supervised model, in addition the unlabeled data collected as described in “A quality metric for heart rate variability from photoplethysmogram sensor data”, of M. Zanon et al., PMID: 33018085, DOI: 10.1109/EMBC44109.2020.9175671 was used. Specifically, in FIGS. 8A to 8C, performance data of the supervised deep learning architecture 134 with and without signal reconstruction, of the semi-supervised deep learning architectures 188 and of a combination 216 of the supervised deep learning architecture 134 with signal reconstruction and semi-supervised deep learning architecture 188 are shown together with a performance of a rescaled HRV multivariate quality metric 218. The performance data for the supervised deep learning architecture 134 are shown for a supervised deep learning architecture 134 trained by optimizing one loss function in terms of classification 220, as exemplarily shown in FIG. 3B, and for a supervised deep learning architecture 134 trained by optimizing two loss functions in terms of signal reconstruction and classification 221, as exemplarily shown in FIG. 3C. The performance data for the semi-supervised deep learning architecture 188 are shown for a semi-supervised deep learning architecture 188 trained by optimizing two loss functions in terms of signal reconstruction and classification, as exemplarily shown in FIG. 5B. The HRV multivariate quality metric may be used for comparison of the deep learning architectures only and is described in further detail in Zanon et al., 2020. Moreover, since the HRV multivariate quality metric refers to a continuous variable with values closer to 0 indicating a clean signal and higher than 20 a noisy signal, a HRV multivariate quality metric 218 rescaled to a range of from 0 to 1 may be used to match the classification output of the different deep learning architectures, where 0 indicates a noisy and 1 a perfectly clean signal. In the diagram of FIG. 8A, the true positive rate 222 of each of the different architectures is shown as a function of the false positive rate 223. FIG. 8B shows the first output 174 for the classified signals using the different architectures, in this example the class output providing the classified quality, together with the corresponding labels 224 of the labelled signals.

Further, FIG. 8C shows accuracies 225 of the different architectures for the specific activities rest begin 202, breathing 204, gaming 206, orthostasis 208, mental stress 210, physical activity 212 and rest end 214. In order to evaluate and compare the performance of the different architectures with each other, one or more of a confusion matrices and/or one or more the following evaluation metrics may be used: accuracy 225; precision; recall; F1-score. Accuracy 225 may be defined as the sum of the true positive results and true negative results divided by the total amount of samples. Precision may refer to the number of true positive results divided by the number of all positive results including those not classified correctly. Recall, also referred to as “else sensitivity”, may be the number of true positive results divided by the number of all samples that should have been identified as positive. The F1-score may refer to the harmonic mean of the precision and recall. The highest possible value of an F1-score may be 1 indicating perfect precision and recall and the lowest possible value may be 0, if either the precision or the recall is zero.

A set of evaluation metrics for the supervised deep learning architecture 134 and the semi-supervised deep learning architecture 188 is shown in Table 1. Specifically, Table 1 shows the results for the supervised deep learning architecture 134 trained by optimizing one loss function in terms of classification 220, as exemplarily shown in FIG. 3B, for the supervised deep learning architecture 134 trained by optimizing two loss functions in terms of signal reconstruction and classification 221, as exemplarily shown in FIG. 3C, once for an equally weighted results and once for optimally weighted results using a weight of 0.1, and similar for the semi-supervised deep learning architecture 188, as exemplarily shown in FIG. 5B, with equally weighted and optimally weighted results, specifically using a weight of 0.05.

As outlined above, when referring to FIGS. 3A to 3C, the class output may contribute to the algorithm learning with a weight of 1. The second output 176 may be weighted using at least one weight. A range of MSE values can be much larger than 1 (which is the maximum class output). Using a weighted second output 176 may allow to balance between the algorithm to learn about the signal reconstruction and about the class output. This may allow increasing the accuracy for both supervised 134 and semi-supervised architectures 188. As can be seen in Table 1, for the supervised architecture 134 accuracy may be 91.6% with equal weights vs 92.5% with lower MSE weight such as a weight of 0.1. For the semi-supervised architecture 188 accuracy may be 87.7% with equal weights vs 90.6% with lower MSE weight such as a weight of 0.05.

TABLE 1
Evaluation metrics for different architectures

| | Accuracy | Precision | F1-score |
|---|---|---|---|
| supervised deep learning | 0.925 | 0.931 | 0.927 |
| architecture 134 trained by | | | |
| optimizing one loss function | | | |
| in terms of classification 220 | | | |
| supervised deep learning | 0.916 | 0.920 | 0.918 |
| architecture 134 trained by | | | |
| optimizing two loss functions | | | |
| in terms of signal reconstruction | | | |
| and classification 221, equal | | | |
| weight | | | |
| supervised deep learning | 0.924 | 0.927 | 0.925 |
| architecture 134 trained by | | | |
| optimizing two loss functions | | | |
| in terms of signal reconstruction | | | |
| and classification 221, optimal | | | |
| weight | | | |
| semi-supervised deep learning | 0.877 | 0.914 | 0.885 |
| architecture 188, equal weight | | | |
| semi-supervised deep learning | 0.906 | 0.923 | 0.920 |
| architecture 188, optimal weight | | | |

The learning rate and decay can vary and this can further increase the accuracy. For example, in case of a learning rate of 0.00001 and with a decay where the learning rate is halved increased from 10 to 100, may result in an increase in accuracy. For example the accuracy may increase by 4.2% for the supervised deep learning architecture 134 trained by optimizing one loss function in terms of classification 220 with equal weights.

The confusion matrices for these architectures are shown in Tables 2 to 5. The columns of the confusion matrices indicate the labeled classification, wherein the rows of the confusion matrices indicate the classified quality obtained by using the trained model 119 with the respective architecture.

TABLE 2
Confusion matrix for the supervised deep learning
architecture 134 trained by optimizing one loss
function in terms of classification 220

| | 0 | 1 |
|---|---|---|
| 0 | 744 | 52 |
| 1 | 23 | 181 |

TABLE 3
Confusion matrix for the supervised deep learning architecture
134 trained by optimizing two loss functions in terms of signal
reconstruction and classification 221, and equal weight

| | 0 | 1 |
|---|---|---|
| 0 | 743 | 53 |
| 1 | 31 | 173 |

TABLE 4
Confusion matrix for the supervised deep learning architecture
134 trained by optimizing two loss functions in terms of signal
reconstruction and classification 221, and optimal weight

| | 0 | 1 |
|---|---|---|
| 0 | 749 | 47 |
| 1 | 29 | 175 |

TABLE 5
Confusion matrix for the semi-supervised deep learning architecture
188 trained by optimizing two loss functions in terms of signal
reconstruction and classification, and equal weight

| | 0 | 1 |
|---|---|---|
| 0 | 682 | 114 |
| 1 | 9 | 195 |

TABLE 6
Confusion matrix for the semi-supervised deep learning architecture
188 trained by optimizing two loss functions in terms of signal
reconstruction and classification, and optimal weight

| | 0 | 1 |
|---|---|---|
| 0 | 718 | 78 |
| 1 | 16 | 188 |

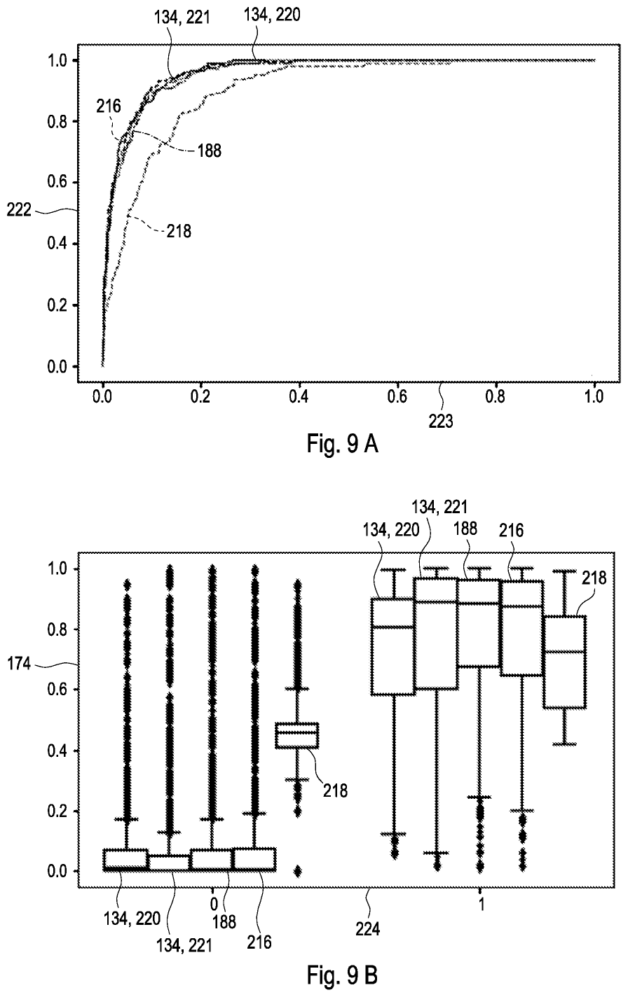

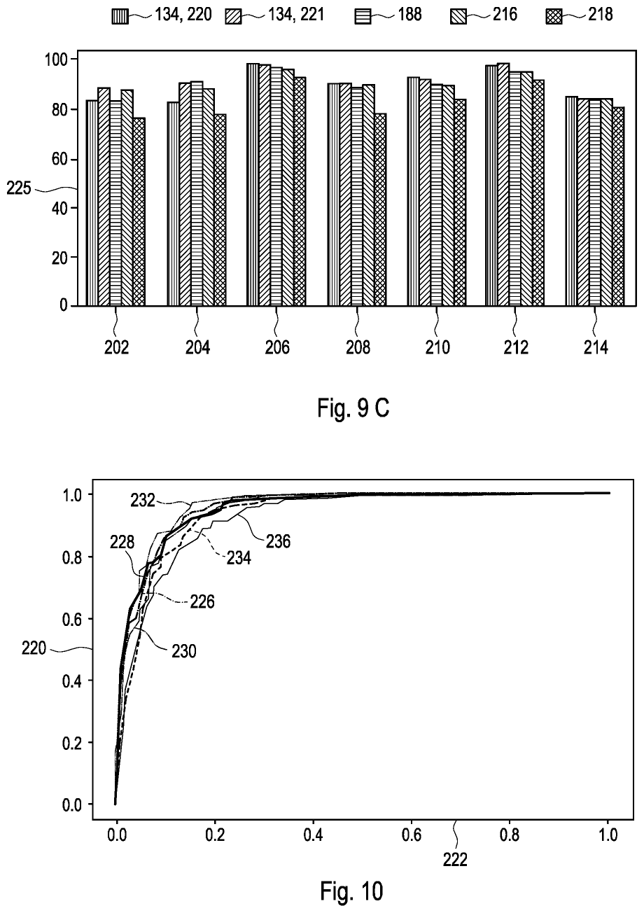

Further, FIGS. 9A to 9C show performance data of different deep learning architectures for a second number of labeled data used for training. The second number of labeled data may comprise 50% of the balanced dataset of 9380 labeled signal samples, i.e. 50% of the first number of labeled data used in FIGS. 8A to 8C. Similar to the previous Figures, FIGS. 9A to 9C show the performance data of the supervised deep learning architecture 134, of the semi-supervised deep learning architectures 188 and of a combination 216 of a supervised 134 and semi-supervised deep learning architecture 188 together with a performance of a rescaled HRV multivariate quality metric 218. The performance data for the supervised deep learning architecture 134 are shown for a supervised deep learning architecture 134 trained by optimizing one loss function in terms of classification 220, as exemplarily shown in FIG. 3B, and for a supervised deep learning architecture 134 trained by optimizing two loss functions in terms of signal reconstruction and classification 221, as exemplarily shown in FIG. 3C. The performance data for the semi-supervised deep learning architecture 188 are shown for a semi-supervised deep learning architecture 188 trained by optimizing two loss functions in terms of signal reconstruction and classification, as exemplarily shown in FIG. 5B.

FIG. 9A shows the true positive rate 222 for each of the different architectures as a function of the false positive rate 223. FIG. 9B shows the first output 174 for the classified signals using the different architectures, in this example the class output providing the classified quality, together with the corresponding labels 224. In FIG. 9C, the accuracies 225 of the different architectures for the specific activities rest begin 202, breathing 204, gaming 206, orthostasis 208, mental stress 210, physical activity 212 and rest end 214 are shown.

In this Example, in which 50% of the training data is used, as outlined above, the set of evaluation metrics for the supervised deep learning architecture 134 and the semi-supervised deep learning architecture 188 is shown in Table 7.

TABLE 7
Evaluation metrics for different architectures

| | Accuracy | Precision | F1-score |
|---|---|---|---|
| supervised deep learning | 0.91 | 0.91 | 0.91 |
| architecture 134 trained | | | |
| by optimizing one loss | | | |
| function in terms of | | | |
| classification 220 | | | |
| supervised deep learning | 0.869 | 0.909 | 0.900 |
| architecture 134 trained | | | |
| by optimizing two loss | | | |
| functions in terms of | | | |
| signal reconstruction and | | | |
| classification 221, equal | | | |
| weight | | | |
| supervised deep learning | 0.905 | 0.908 | 0.906 |
| architecture 134 trained | | | |
| by optimizing two loss | | | |
| functions in terms of | | | |
| signal reconstruction and | | | |
| classification 221, optimal | | | |
| weight | | | |
| semi-supervised deep | 0.861 | 0.881 | 0.867 |
| learning architecture | | | |
| 188, equal weight | | | |
| semi-supervised deep | 0.906 | 0.915 | 0.909 |
| learning architecture | | | |
| 188, optimal weight | | | |

The confusion matrices for these architectures are shown in Tables 8 to 12.

TABLE 8
Confusion matrix for the supervised deep learning
architecture 134 trained by optimizing one loss
function in terms of classification 220

| | 0 | 1 |
|---|---|---|
| 0 | 751 | 45 |
| 1 | 45 | 159 |

TABLE 9
Confusion matrix for the supervised deep learning architecture
134 trained by optimizing two loss functions in terms of signal
reconstruction and classification 221, and equal weight

| | 0 | 1 |
|---|---|---|
| 0 | 720 | 76 |
| 1 | 28 | 176 |

TABLE 10
Confusion matrix for the supervised deep learning architecture
134 trained by optimizing two loss functions in terms of signal
reconstruction and classification 221, and optimal weight

| | 0 | 1 |
|---|---|---|
| 0 | 740 | 56 |
| 1 | 39 | 165 |

TABLE 11
Confusion matrix for the semi-supervised deep learning architecture
188 trained by optimizing two loss functions in terms of signal
reconstruction and classification, and equal weight

| | 0 | 1 |
|---|---|---|
| 0 | 696 | 100 |
| 1 | 39 | 165 |

TABLE 12
Confusion matrix for the semi-supervised deep learning architecture
188 trained by optimizing two loss functions in terms of signal
reconstruction and classification, and optimal weight

| | 0 | 1 |
|---|---|---|
| 0 | 730 | 66 |
| 1 | 28 | 176 |

Using the semi-supervised architecture 188 may allow to expand and/or transfer the proposed model to new dataset. For example, if there is a need to adapt the model to a new scenario, even with less data, such as up to less than 50% of the data used for training the originally trained model, e.g. because there are not enough labeled examples, it is possible to train a reliable model using the semi-supervised approach. It was found that accuracy remains >90% with all algorithms when optimal weight for the reconstruction loss is applied.

In FIG. 10, performance data of the semi-supervised deep learning architecture 188 for different shares of labeled signals are shown. The semi-supervised deep learning architecture 188 may be embodied as exemplarily shown in FIG. 5A. The performance of the semi-supervised deep learning architecture 188 is shown for a share of 100% of labeled data of the labeled dataset (denoted by reference number 226), for a share of 90% of labeled data of the labeled dataset (denoted by reference number 228), for a share of 75% of labeled data of the labeled dataset (denoted by reference number 230), for a share of 50% of labeled data of the labeled dataset (denoted by reference number 232), for a share of 25% of labeled data of the labeled dataset (denoted by reference number 234) and for a share of 10% of labeled data of the labeled dataset (denoted by reference number 236). The performance data shown in FIG. 10 were obtained for equally weighted loss functions, specifically for a weight of 1 for both the first loss function, i.e. the classification loss function, and the second loss function, i.e. the signal reconstruction loss function.

As can be seen in FIG. 10, the performance of the semi-supervised deep learning architecture 188 of FIG. 5A may slowly decrease as the share of labeled data is lowered. Using 50% of labeled data may leave more room for the unlabeled data to contribute, thus, showing an increased area under the curve (AUC) compared when using a share of 100% of the available labeled data.

### LIST OF REFERENCE NUMBERS

110 biological sensor data
112 biological sensor
114 measuring unit
116 signal
118 processing unit
119 trainable model
120 portable photoplethysmogram device
122 illumination source
124 photodetector
126 photoplethysmogram
128 providing biological sensor data
130 classifying quality of the signal
131 pre-processing step
132 training step
134 supervised deep learning architecture
136 input layer
138 input of the input layer
140 plurality of convolutional layers
142 stack of convolutional layers
144 first convolutional layer
146 second convolutional layer
148 third convolutional layer
150 fourth convolutional layer
152 fifth convolutional layer
154 output of the input layer
156 output of the first convolutional layer
158 output of the second convolutional layer
160 output of the third convolutional layer
162 output of the fourth convolutional layer
164 flatten layer
166 matrix output of the fifth convolutional layer
168 dense layer
170 output layer
172 output of the flatten layer
174 first output
176 second output
178 first extra dense layer
180 second extra dense layer
182 output of the first extra dense layer
184 reconstructed signal
186 original signal
188 semi-supervised deep learning architecture
190 extra input parameter zᵢₙₚᵤₜ
192 additional input layer
194 output of the additional input layer
196 additional output layer
198 resulting output
200 amount of data points
202 rest begin
204 breathing
206 gaming
208 orthostasis
210 mental stress
212 physical activity
214 rest end
216 combination of a supervised and semi-supervised deep learning architecture with signal reconstruction
218 rescaled HRV multivariate quality metric
220 supervised deep learning architecture trained by optimizing one loss function in terms of classification
221 supervised deep learning architecture trained by optimizing two loss functions in terms of signal reconstruction and classification
222 true positive rate
223 false positive rate
224 label
225 accuracy
226 semi-supervised deep learning architecture for a share of 100% of labeled data
228 semi-supervised deep learning architecture for a share of 90% of labeled data
230 semi-supervised deep learning architecture for a share of 75% of labeled data
232 semi-supervised deep learning architecture for a share of 50% of labeled data
234 semi-supervised deep learning architecture for a share of 25% of labeled data
236 semi-supervised deep learning architecture for a share of 10% of labeled data

1. A computer-implemented method for classifying quality of biological sensor data comprising:
providing biological sensor data obtained by at least one biological sensor, wherein the biological sensor data comprises at least one signal; and
classifying a quality of the at least one signal by using a trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.

2. The method of claim 1, wherein the at least one biological sensor is at least one portable photoplethysmogram device and the biological sensor data comprises at least one photoplethysmogram obtained by the at least one portable photoplethysmogram device.

3. The method of claim 2, wherein the quality of the at least one signal is used as a quality indicator for heart rate variability data, wherein the quality is used for distinguishing between acceptable and non-acceptable heart rate variability data.

4. The method of claim 1, wherein classifying the quality of the at least one signal comprises discriminating between noisy and clean signals.

5. The method of claim 1, wherein the trainable model comprises at least one deep neural network selected from the group consisting of a Convolutional Neural Network (CNN), a recurrent neural network (RNN), and a Long short-term memory (LSTM).

6. The method of claim 1, further comprising training the trainable model on the at least one training dataset comprising the historical biological sensor data, based on the supervised and/or semi-supervised deep learning architecture.

7. The method of claim 6, wherein the historical biological sensor data comprises manually labeled historical biological sensor data; and
wherein training the trainable model comprises training the trainable model on the at least one dataset comprising the manually labeled historical biological sensor data based on the supervised deep learning architecture.

8. The method of claim 6, wherein the historical biological sensor data comprises manually labeled historical biological sensor data and unlabeled historical biological sensor data; and
wherein training the trainable model comprises training the trainable model on the at least one dataset comprising the manually labeled and the unlabeled historical biological sensor data based on the semi-supervised deep learning architecture.

9. The method of claim 8, wherein for the unlabeled historical biological sensor data, the trainable model is trained by optimizing the loss function in terms of signal reconstruction and by disregarding the loss function in terms of classification.

10. The method of claim 1, further comprising preprocessing the biological sensor data by one or more of filtering or normalizing the biological sensor data.

11. A biological sensor for classifying quality of biological sensor data, wherein the biological sensor comprises:
at least one measuring unit configured to generate biological sensor data comprising at least one signal;
at least one processing unit; and
a computer-readable storage medium comprising instructions stored thereon which, when executed by the at least one processing unit, causes the biological sensor to:
classify a quality of the at least one signal by using a trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.

12. The biological sensor of claim 11, wherein the biological sensor is a portable photoplethysmogram device and further comprises:
at least one illumination source;
at least one photodetector, wherein the at least one illumination source and the at least one photodetector are configured to provide at least one photoplethysmogram; and
wherein the instructions further cause the biological sensor to classify a quality of the photoplethysmogram with the trainable model.

13-15. (canceled)

16. The biological sensor of claim 11, wherein to classify the quality of the at least one signal comprises discriminating between noisy and clean signals.

17. The biological sensor of claim 11, wherein the trainable model is trained with labeled historical biological sensor data and unlabeled historical biological sensor data based on the semi-supervised deep learning architecture.

18. The biological sensor of claim 11, wherein the instructions further cause the biological sensor to one or more of filter or normalize the biological sensor data generated by the at least one measuring unit.

19. A computer-readable storage medium comprising instructions stored thereon that, in response to execution by a processor, causes a biological sensor to:
generate biological sensor data comprising at least one signal; and
classify a quality of the at least one signal by using a trainable model, wherein the trainable model is trained on historical biological sensor data based on a supervised and/or semi-supervised deep learning architecture, wherein the trainable model is trained by optimizing one loss function in terms of classification or two loss functions in terms of signal reconstruction and classification.

20. The computer-readable storage medium of claim 19, wherein the biological sensor is a portable photoplethysmogram device and the biological sensor data comprises at least one photoplethysmogram obtained by the at least one portable photoplethysmogram device.

21. The computer-readable storage medium of claim 19, wherein to classify a quality of the at least one signal by using a trainable model comprises to discriminate between noisy signals and clean signals.

22. The computer-readable storage medium of claim 19, wherein the trainable model is trained with labeled historical biological sensor data and unlabeled historical biological sensor data based on the semi-supervised deep learning architecture.

23. The computer-readable storage medium of claim 19, wherein the instructions further cause the biological sensor to one or more of filter or normalize the generated biological sensor data.
